(19) United States
(12) Patent Application Publication
(10) Pub. No.: US 2025/0136763 A1
(43) Pub. Date: May 1, 2025

(54) SILICONE - VINYLESTER FUNCTIONAL COMPOUNDS AND METHODS FOR THEIR PREPARATION AND USE IN PERSONAL CARE COMPOSITIONS

(71) Applicants: Dow Silicones Corporation, Midland, MI (US); Dow Global Technologies LLC, Midland, MI (US); Rohm and Haas Company, Collegeville, PA (US)

(72) Inventors: Ligeng Yin, Collegeville, PA (US); Nanguo Liu, Midland, MI (US); Erich Molitor, Midland, MI (US); Haoquan Li, Midland, MI (US); Jason Fisk, Midland, MI (US); Michaeleen Pacholski, Collegeville, PA (US); Xiaodong Lu, Collegeville, PA (US); Matthew Carter, Collegeville, PA (US); Liyi Tan, Ichihara (JP); Tian Lan, Collegeville, PA (US)

(21) Appl. No.: 18/836,784

(22) PCT Filed: Mar. 13, 2023
(86) PCT No.: PCT/US2023/064208

§ 371 Date: Aug. 8, 2024

Related U.S. Application Data
(60) Provisional application No. 63/330,511, filed on Apr. 13, 2022.

(51) Int. Cl. C08G 77/04; A61K 8/895; A61Q 1/02; A61Q 17/04; C08F 290/06; C08G 77/00; C08G 77/08; C08G 77/32; C08G 77/442

(52) U.S. Cl. CPC C08G 77/045; A61K 8/895; A61Q 1/02; A61Q 17/04; C08F 290/068; C08G 77/08; C08G 77/32; C08G 77/442; C08G 77/70; A61K 2800/95

(57) ABSTRACT

A silicone-vinylester copolymer and methods for its preparation and use are provided. The silicone-vinylester copolymer can be made using a vinylester-functional siloxane macromonomer, which can be prepared by a process comprising hydroformylation of an alkenyl-functional polyorganosiloxane to form an aldehyde-functional polyorganosiloxane; oxidation of the aldehyde-functional polyorganosiloxane to form a carboxy-functional polyorganosiloxane, and transvinylation of the carboxy-functional polyorganosiloxane to form the vinylester-functional siloxane macromonomer. The silicone-vinylester copolymer is useful in personal care compositions.

### CROSS REFERENCE TO RELATED APPLICATIONS

This application claims the benefit of U.S. Provisional Patent Application Ser. No. 63/330,511 filed on 13 Apr. 2022 under 35 U.S.C. § 119(e). U.S. Provisional Patent Application Ser. No. 63/330,511 is hereby incorporated by reference.

### TECHNICAL FIELD

This invention relates to a vinylester-functional siloxane macromonomer (macromonomer) that can be copolymerized with a vinylester of an aliphatic fatty acid to form a silicone-vinylester copolymer (copolymer) and methods for the preparation of the macromonomer and copolymer. The copolymer may be added to personal care compositions suitable for application to human skin.

### INTRODUCTION

Film forming agents are important cosmetic raw ingredients, and more often than not, broadly used in personal care compositions, e.g., leave-on products applied to human skin, such as skin care, sunscreen, and color cosmetic products. The DOWSIL™ silicone acrylate FA series products such as DOWSIL™ FA 4004 and DOWSIL™ FA 4012 offered by Dow Silicones Corporation of Midland, Michigan, USA, has been used as film forming agents in personal care compositions. However, there are ongoing needs in the cosmetics industry for sustainable and/or naturally derived ingredients that have one or more of the following properties: biodegradability potential, water resistance, sebum resistance, rub off resistance, and favorable sensory properties.

### SUMMARY

A vinylester-functional siloxane macromonomer (macromonomer) and silicone-vinylester copolymer (copolymer) are disclosed. Methods for the preparation of the macromonomer and the copolymer are provided. The copolymer is useful in a personal care composition.

### DETAILED DESCRIPTION

This copolymer may be obtained by a process comprising copolymerizing a mixture of monomers comprising: M1) the vinylester-functional siloxane macromonomer (introduced above) and M2) a vinyl ester of an aliphatic fatty acid. The copolymer can be used in a personal care composition.

### Starting Material M1) Vinylester-Functional Siloxane Macromonomer

Starting material M1) is the vinylester-functional siloxane macromonomer (macromonomer) introduced above. The amount of M1) the macromonomer in the mixture of monomers depends on various factors including the selection and amount of other monomers in the mixture and on the desired end use of the copolymer. However, the amount of M1) the macromonomer may be 1% to 99%, alternatively 30% to 60%, based on combined weights of all monomers in the mixture of monomers (e.g., based on combined weights of M1), M2), and M3), described herein).

The macromonomer may have formula (M1-1): 0

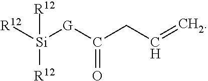

where G is a divalent hydrocarbon group free of aliphatic unsaturation, each R¹² is independently selected from —OSi(R¹⁴)₃ and R¹³, where each R¹³ is a monovalent hydrocarbon group; where each R¹⁴ is selected from R¹³, —OSi(R¹⁵)₃, and —[OSiR¹³₂]iᵢOSiR¹³₃; where each R¹⁵ is selected from R¹³, —OSi(R¹⁶)₃, and —[OSiR¹³₂]ᵢᵢOSiR¹³₃; where each R¹⁶ is selected from R¹³ and —[OSiR¹³₂]ᵢᵢOSiR¹³₃; where each subscript ii independently has a value such that 0≤ii≤100, with the proviso that at least two of R¹² are —OSi(R¹⁴)₃ and the macromonomer has 4 to 16 silicon atoms per molecule. Alternatively, each R¹³ may be an independently selected alkyl group. Alternatively, each R¹³ may be methyl. At least two of R¹² may be —OSi(R¹⁴)₃. Alternatively, all three of R¹² may be —OSi(R¹⁴)₃.

Examples of divalent hydrocarbon groups for G include alkylene groups of empirical formula —CᵣH₂ᵣ—, where subscript r is 2 to 8. The alkylene group may be a linear alkylene, e.g., —CH₂—CH₂—, —CH₂—CH₂—CH₂—, —CH₂—CH₂—CH₂—CH₂—, or —CH₂—CH₂—CH₂—CH₂—CH₂—CH₂—, or a branched alkylene, e.g.,

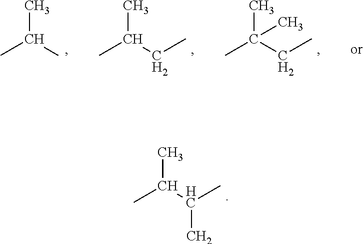

Alternatively, the divalent hydrocarbon group for G may be an arylene group such as phenylene, or an alkylarylene group such as: H or

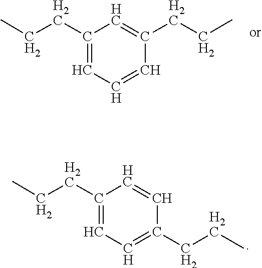

Alternatively, G may be the alkylene group, such as ethylene.

Each R¹³ is an independently selected monovalent hydrocarbon group. The monovalent hydrocarbon group for R¹³ may be independently selected from the group consisting of an alkyl group of 1 to 18 carbon atoms and an aryl group of 6 to 18 carbon atoms. Suitable alkyl groups for R¹³ may be linear, branched, cyclic, or combinations of two or more thereof. The alkyl groups are exemplified by methyl, ethyl, propyl (including n-propyl and/or isopropyl), butyl (including n-butyl, tert-butyl, sec-butyl, and/or isobutyl); pentyl, hexyl, heptyl, octyl, decyl, dodecyl, undecyl, and octadecyl (and saturated, branched isomers having 5 to 18 carbon atoms), and the alkyl groups are further exemplified by cycloalkyl groups such as cyclopropyl, cyclobutyl, cyclopentyl, and cyclohexyl. Alternatively, the alkyl group for R¹³ may be selected from the group consisting of methyl, ethyl, propyl and butyl; alternatively methyl, ethyl, and propyl; alternatively methyl and ethyl. Alternatively, the alkyl group for R¹³ may be methyl.

Suitable aryl groups for R¹³ may be monocyclic or polycyclic and may have pendant hydrocarbyl groups. For example, the aryl groups for R¹³ include phenyl, tolyl, xylyl, and naphthyl and further include aralkyl groups such as benzyl, 1-phenylethyl, and 2-phenylethyl. Alternatively, the aryl group for R¹³ may be monocyclic, such as phenyl, tolyl, or benzyl; alternatively the aryl group for R¹³ may be phenyl.

Alternatively, when in formula (M1-1) each R¹² is —OSi(R¹⁴)₃ and each R¹⁴ is —OSi(R¹⁵)₃, the macromonomer may have structure (M1-2):

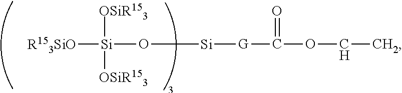

where G and R¹⁵ are as described above. Alternatively, in this structure each R¹⁵ may be an R¹³, as described above, and each R¹³ may be methyl.

Alternatively, in formula (M1-1), when each R¹² is —OSi(R¹⁴)₃, one R¹⁴ may be R¹³ in each —OSi(R¹⁴)₃ such that each R¹² is —OSiR¹³(R¹⁴)₂. Alternatively, two R¹⁴ in —OSiR¹³(R¹⁴)₂ may each be —OSi(R¹⁵)₃ moieties such that the macromonomer has the following structure (M1-3):

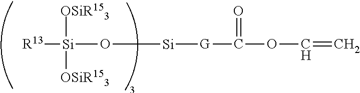

where G, R¹³, and R¹⁵ are as described above. Alternatively, in this structure, each R¹⁵ may be an R¹³, as described above, and each R¹³ may be methyl.

Alternatively, in formula (M1-1), one R may be R¹³, and two of R¹² may be —OSi(R¹⁴)₃. When two of R¹² are —OSi(R¹⁴)₃, and one R¹⁴ is R¹³ in each —OSi(R¹⁴)₃ then two of R¹² are —OSiR¹³(R¹⁴)₂. Alternatively, each R¹⁴ in —OSiR³(R¹⁴)₂ may be —OSi(R¹⁵)₃ such that the macromonomer has the following structure (M1-4):

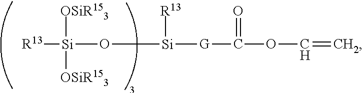

where G, R¹³, and R¹⁵ are as described above. Alternatively, in this formula, each R⁵ may be an R¹³, and each R¹³ may be methyl.

Examples of the macromonomer include (M1-5): vinyl 3-(1,1,1,5,5,5-hexamethyl-3-((trimethylsilyl)oxy)trisiloxan-3-yl)propanoate, which has formula (M1-6):

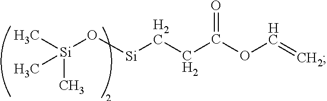

vinyl 3-(1,1,1,3,5,7,9,9,9-nonamethyl-37-bis((trimethylsilyl)oxy)pentasiloxan-5-yl)propanoate, which has formula (M1-7):

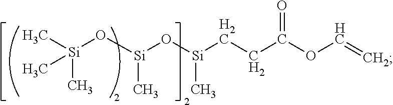

vinyl 3-(5-((1,1,1,3,5,5,5-heptamethyltrisiloxan-3-yl)oxy)-1,1,1,3,7,9,9,9-octamethyl-3,7-bis((trimethylsilyl)oxy)pentasiloxan-5-yl)propanoate, which has formula (M1-8):

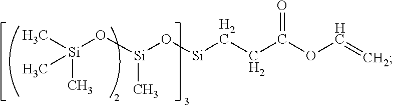

and
vinyl 7-(5-((1,1,1,3,5,5,5-heptamethyltrisiloxan-3-yl)oxy)-1,1,1,3,7,9,9,9-octamethyl-3,7-bis((trimethylsilyl)oxy)pentasiloxan-5-yl)heptanoate, which has formula

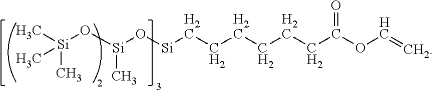

Alternatively, starting material M1) the macromonomer may be linear. Alternatively, the macromonomer may comprise (M1-9), a linear polydiorganosiloxane having, per molecule, at least one vinylester-functional group; alternatively at least two vinylester-functional groups. For example, said polydiorganosiloxane may comprise unit formula (M1-10): (R⁴₃SiO_{1/2})ₐ(R^{VF}R⁴₂SiO_{1/2})_{b}(R⁴₂SiO_{2/2})_{c}(R^{VE}R⁴SiO_{2/2})_{d}, where R^{VE} is

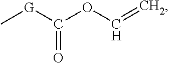

where G is a divalent hydrocarbon group free of aliphatic unsaturation as described above for formula (M1-1), each R⁴ is independently selected from the group consisting of an alkyl group of 1 to 18 carbon atoms and an aryl group of 6 to 18 carbon atoms, and subscripts a, b, c, and d represent average numbers of each unit in the formula and have values such that subscript a is 0, 1, or 2; subscript b is 0, 1, or 2, subscript c≥0, subscript d≥0, with the provisos that a quantity (b+d)≥1, a quantity (a+b)=2, and a quantity (a+b+c+d)≥2. The quantity (a+b+c+d) may be at least 3, alternatively at least 4, and alternatively ≥50. At the same time in unit formula (M1-1), the quantity (a+b+c+d) may be less than or equal to 10,000; alternatively less than or equal to 4,000; alternatively less than or equal to 2,000; alternatively less than or equal to 1,000; alternatively less than or equal to 500; alternatively less than or equal to 250.

Suitable alkyl groups for R⁴ may be linear, branched, cyclic, or combinations of two or more thereof. The alkyl groups are exemplified by methyl, ethyl, propyl (including n-propyl and/or isopropyl), butyl (including n-butyl, tert-butyl, sec-butyl, and/or isobutyl); pentyl, hexyl, heptyl, octyl, decyl, dodecyl, undecyl, and octadecyl (and branched isomers having 5 to 18 carbon atoms), and the alkyl groups are further exemplified by cycloalkyl groups such as cyclopropyl, cyclobutyl, cyclopentyl, and cyclohexyl. Alternatively, the alkyl group for R⁴ may be selected from the group consisting of methyl, ethyl, propyl and butyl; alternatively methyl, ethyl, and propyl; alternatively methyl and ethyl. Alternatively, the alkyl group for R⁴ may be methyl.

Suitable aryl groups for R⁴ may be monocyclic or polycyclic and may have pendant hydrocarbyl groups. For example, the aryl groups for R⁴ include phenyl, tolyl, xylyl, and naphthyl and further include aralkyl groups such as benzyl, 1-phenylethyl and 2-phenylethyl. Alternatively, the aryl group for R⁴ may be monocyclic, such as phenyl, tolyl, or benzyl; alternatively the aryl group for R⁴ may be phenyl.

Alternatively, the linear vinylester-functional polydiorganosiloxane of unit formula (M1-10) may be selected from the group consisting of: unit formula (M1-11): (R⁴₂R^{VE}SiO_{1/2})₂(R⁴₂SiO_{2/2})ₘ(R⁴R^{VE}SiO_{2/2})ₙ, unit formula (M1-12): (R⁴₃SiO_{1/2})₂(R⁴₂SiO_{2/2})ₒ(R⁴R^{VE}SiO_{2/2})ₚ, or a combination of both (M1-11) and (M1-12). In formulae (M1-11) and (M1-12), each R^{VE} and each R⁴ are as described above for formula (M1-10), and subscripts m, n, o, and p represent average numbers of each unit in unit formulas (M1-11) and (M1-12). Subscripts m, n, o, and p have the following values: Subscript m may be 0 or a positive number. Alternatively, subscript m may be at least 2. Alternatively subscript m be 2 to 2,000. Subscript n may be 0 or a positive number. Alternatively, subscript n may be 0 to 2000. Subscript o may be 0 or a positive number. Alternatively, subscript o may be 0 to 2000. Subscript p is at least 2.

Alternatively subscript p may be 2 to 2000.

Alternatively, the polydiorganosiloxane of unit formula (M1-10) may have formula (M1-13):

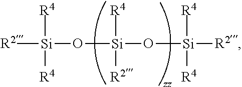

where each R^{2′″} is independently selected from the group consisting of R⁴ and R^{VE}, with the proviso that at least one R^{2′″} per molecule is R^{VE}, each R⁴ and each R^{VE} is as described above for formula (M1-10), and subscript zz=a quantity (c+d). Alternatively, subscript zz may have a value of 0 to 2,000; alternatively 0 to 1,000; alternatively 50 to 2,000; alternatively 50 to 1,000; and alternatively 80 to 800.

Alternatively, the polydiorganosiloxane of formula (M1-10) may comprise two different endgroups, i.e., when subscript a=1 and subscript b=1. Alternatively, this polydiorganosiloxane may be monofunctional, having one vinylester-functional group per molecule, e.g., when subscript a =1, b=1, and d=0 in unit formula (M1-10). This polydiorganosiloxane may have formula (M1-14):

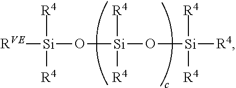

where each R⁴ and each R^{VE} and subscript c are as described above for formula (M1-1). Alternatively, in formula (M1-14), subscript c may have a value of 0 to 2,000; alternatively 0 to 1,000; alternatively 50 to 2,000; alternatively 50 to 1,000; and alternatively 80 to 800.

Starting material M1) the macromonomer may comprise a vinylester-functional polydiorganosiloxane such as i) bis-dimethyl(propanoate)siloxy-terminated polydimethylsiloxane, ii) bis-dimethyl(propanoate)siloxy-terminated poly(dimethylsiloxane/methyl(propanoate)siloxane), iii) bis-dimethyl(propanoate)siloxy-terminated polymethyl(propanoate)siloxane, iv) bis-trimethylsiloxy-terminated poly(dimethylsiloxane/methyl(propanoate)siloxane), v) bis-trimethylsiloxy-terminated polymethyl(propanoate)siloxane, vi) bis-dimethyl(propanoate)siloxy-terminated poly(dimethylsiloxane/methylphenylsiloxane/methyl(propanoate)siloxane), vii) bis-dimethyl(propanoate)siloxy-terminated poly(dimethylsiloxane/methylphenylsiloxane), viii) bis-dimethyl(propanoate)siloxy-terminated poly(dimethylsiloxane/diphenylsiloxane), ix) bis-phenyl,methyl,(propanoate)-siloxy-terminated polydimethylsiloxane, x) bis-dimethyl(heptanoate)siloxy-terminated polydimethylsiloxane, xi) bis-dimethyl(heptanoate)siloxy-terminated poly(dimethylsiloxane/methyl(heptanoate)siloxane), xii) bis-dimethyl(heptanoate)siloxy-terminated polymethyl(heptanoate)siloxane, xiii) bis-trimethylsiloxy-terminated poly(dimethylsiloxane/methyl(heptanoate)siloxane), xiv) bis-trimethylsiloxy-terminated polymethyl(heptanoate)siloxane, xv) bis-dimethyl(heptanoate)-siloxy terminated poly(dimethylsiloxane/methylphenylsiloxane/methyl(heptanoate)siloxane), xvi) bis-dimethyl(propanoate)siloxy-terminated poly(dimethylsiloxane/methyl(heptanoate)siloxane), xvii) bis-dimethyl(heptanoate)-siloxy-terminated poly(dimethylsiloxane/methylphenylsiloxane), xviii) bis-dimethyl(heptanoate)-siloxy-terminated poly(dimethylsiloxane/diphenylsiloxane), xix) alpha-dimethyl(n-butyl)siloxy-omega-dimethyl(propanoate)siloxy-terminated poly(dimethylsiloxane), and xx) a combination of two or more of i) to xix).

### Process to Make the Vinylester-Functional Siloxane Macromonomer

The macromonomer described above may be prepared by a transvinylation reaction process comprising:
optionally 1) combining, under conditions to catalyze a hydroformylation reaction, starting materials comprising (A) a gas comprising hydrogen and carbon monoxide, (B) an alkenyl-functional polyorganosiloxane, (C) a rhodium/bisphosphite ligand complex catalyst, and optionally (D) a solvent; thereby forming (E) an aldehyde-functional polyorganosiloxane;
optionally 2) recovering (E) the aldehyde-functional polyorganosiloxane;
optionally 3) combining, under conditions to conduct an oxidation reaction, starting materials comprising (E) the aldehyde-functional polyorganosiloxane, (F) an oxygen source, optionally (G) an oxidation reaction catalyst, and optionally (H) a second solvent; thereby forming an oxidation reaction product comprising (I) a carboxy-functional polyorganosiloxane;
optionally 4) recovering (I) the carboxy-functional polyorganosiloxane;
5) combining, under conditions to conduct a transvinylation reaction, starting materials comprising (I) the carboxy-functional polyorganosiloxane, (J) an alkenyl acetate, (K) a transvinylation catalyst, and optionally (L) a third solvent, and optionally (X) an inhibitor, thereby producing a transvinylation reaction product comprising (M1) the macromonomer described above; and optionally 6) recovering (M1) the macromonomer.

### Hydroformylation

In the transvinylation reaction process for making the macromonomer described herein, a process comprising hydrofonnylation of an alkenyl-functional polyorganosiloxane to form an aldehyde-functional polyorganosiloxane and subsequent oxidation of the aldehyde-functional polyorganosiloxane to form starting material (I), the carboxy-functional polyorganosiloxane, may be performed. The hydroformylation reaction in step 1) may be performed at relatively low temperature. For example, the hydroformylation reaction in step 1) may be performed at a temperature of at least 30° C., alternatively at least 50° C., and alternatively at least 70° C. At the same time, the temperature for the hydroformylation reaction may be up to 150° C.; alternatively up to 100° C.; alternatively up to 90° C., and alternatively up to 80° C. Without wishing to be bound by theory, it is thought that lower temperatures, e.g., 30° C. to 90° C., alternatively 40° C. to 90° C., alternatively 50° C. to 90° C., alternatively 60° C. to 90° C., alternatively 70° C. to 90° C., alternatively 80° C. to 90° C., alternatively 30° C. to 60° C., alternatively 50° C. to 60° C., may be desired for achieving high selectivity and ligand stability.

In the process described herein, hydroformylation reaction in step 1) may be performed at a pressure of at least 101 kPa (ambient), alternatively at least 206 kPa (30 psi), and alternatively at least 344 kPa (50 psi). At the same time, pressure in step 1) may be up to 6,895 kPa (1,000 psi), alternatively up to 1,379 kPa (200 psi), alternatively up to 1000 kPa (145 psi), and alternatively up to 689 kPa (100 psi). Alternatively, step 1) may be performed at 101 kPa to 6,895 kPa; alternatively 344 kPa to 1,379 kPa; alternatively 101 kPa to 1,000 kPa; and alternatively 344 kPa to 689 kPa. Without wishing to be bound by theory, it is thought that using relatively low pressures, e.g., <6,895 kPa in the hydroformylation reaction step of the process herein may be beneficial; the ligands described herein allow for low pressure hydroformylation reactions, which have the benefits of lower cost and better safety than high pressure hydroformylation reactions.

The hydroformylation reaction step of the process may be carried out in a batch, semi-batch, or continuous mode, using one or more suitable reactors, such as a fixed bed reactor, a fluid bed reactor, a continuous stirred tank reactor (CSTR), or a slurry reactor. The selection of (B) the alkenyl-functional polyorganosiloxane, and (C) the catalyst, and whether (D) the solvent, is used may impact the size and type of reactor used. One reactor, or two or more different reactors, may be used. The hydroformylation process may be conducted in one or more steps, which may be affected by balancing capital costs and achieving high catalyst selectivity, activity, lifetime, and ease of operability, as well as the reactivity of the particular starting materials and reaction conditions selected, and the desired product.

Alternatively, the hydroformylation reaction may be performed in a continuous manner.

For example, the hydroformylation reaction step of the process used may be as described in U.S. Pat. No. 10,023,516 except that the olefin feed stream and catalyst described therein are replaced with (B) the alkenyl-functional polyorganosiloxane and (C) the rhodium/bisphosphite ligand complex catalyst, each described herein.

Step 1) of the process forms a hydroformylation reaction product comprising (E) the aldehyde-functional polyorganosiloxane. The hydroformylation reaction product may further comprise additional materials, such as those which have either been deliberately employed, or formed in situ, during step 1) of the process. Examples of such materials that can also be present include unreacted (B) alkenyl-functional polyorganosiloxane, unreacted (A) carbon monoxide and hydrogen gases, and/or in situ formed side products, such as ligand degradation products and adducts thereof, and high boiling liquid aldehyde condensation byproducts, as well as (D) a solvent, if employed. The term “ligand degradation product” includes but is not limited to any and all compounds resulting from one or more chemical transformations of at least one of the ligand molecules used in the process.

The process may further comprise an additional step such as: 2) recovering (E) the aldehyde-functional polyorganosiloxane from the hydroformylation reaction product. This may be performed by separating (C) the rhodium/bisphosphite ligand complex catalyst from the hydroformylation reaction product. Separating (C) the rhodium/bisphosphite ligand complex catalyst may be performed by methods known in the art, including but not limited to adsorption and/or membrane separation (e.g., nanofiltration). Suitable recovery methods are as described, for example, in U.S. Pat. No. 5,681,473 to Miller, et al.; U.S. Pat. No. 8,748,643 to Priske, et al.; and 10,155,200 to Geilen, et al.

However, one benefit of the process described herein is that (C) the hydroformylation reaction catalyst need not be removed and recycled. Due to the low level of Rh needed, it may be more cost effective not to recover and recycle (C) the hydroformylation reaction catalyst; and the aldehyde-functional polyorganosiloxane produced by the process may be stable even when the hydroformylation reaction catalyst is not removed. Furthermore, without wishing to be bound by theory, it is thought that (C) the hydroformylation reaction catalyst may also catalyze the oxidation reaction of the aldehyde-functional polyorganosiloxane, as described herein below. Therefore, alternatively, the hydroformylation process described above may be performed without removal of the hydroformylation reaction catalyst in step 2).

Alternatively, optional step 2) of the process may comprise purification of the hydroformylation reaction product. For example, the aldehyde-functional polyorganosiloxane may be isolated from the additional materials, described above, by any convenient means such as stripping and/or distillation, optionally with reduced pressure. Alternatively, step 2) may be omitted, for example, to leave (C) the hydroformylation reaction catalyst in the hydroformylation reaction product comprising the aldehyde-functional polyorganosiloxane.

### (A) Syngas

Starting material (A), the gas used in the hydroformylation process, comprises carbon monoxide (CO) and hydrogen gas (H₂). For example, the gas may be syngas. As used herein, “syngas” (from synthesis gas) refers to a gas mixture that contains varying amounts of CO and H₂.

Production methods are well known and include, for example: (1) steam reforming and partial oxidation of natural gas or liquid hydrocarbons, and (2) the gasification of coal and/or biomass. CO and H₂ typically are the main components of syngas, but syngas may contain carbon dioxide and inert gases such as CH₄, N₂ and Ar. The molar ratio of H₂ to CO (H₂:CO molar ratio) varies greatly but may range from 1:100 to 100:1, alternatively 1:10 and 10:1. Syngas is commercially available and is often used as a fuel source or as an intermediate for the production of other chemicals.

Alternatively, CO and H₂ from other sources (i.e., other than syngas) may be used as starting material (A) herein. Alternatively, the H₂:CO molar ratio in starting material (A) for use herein may be 3:1 to 1:3, alternatively 2:1 to 1:2, and alternatively 1:1.
(B) Alkenyl-functional polyorganosiloxane

Starting material (B) used in the process described herein is an alkenyl-functional polyorganosiloxane. The alkenyl-functional polyorganosiloxane may be branched. The branched alkenyl-functional polyorganosiloxane may have general formula (B1-1): R^{A}SiR¹²₃, where R^{A} is an alkenyl group, and each R¹² is selected from —OSi(R¹⁴)₃ and R¹³; where each R¹³ is a monovalent hydrocarbon group; where each R¹⁴ is selected from R¹³, —OSi(R¹⁵)₃, and —[OSiR¹³₂]ᵢᵢOSiR¹³₃; where each R¹⁵ is selected from R¹³, —OSi(R¹⁶)₃, and —[OSiR¹³₂]ᵢᵢOSiR¹³₃; where each R¹⁶ is selected from R¹³ and —[OSiR¹³₂]ᵢᵢOSiR¹³₃; and where subscript ii has a value such that 0≤ii≤100. At least two of R¹² may be —OSi(R¹⁴)₃. Alternatively, all three of R¹² may be —OSi(R¹⁴)₃.

Each R^{A} is an independently selected alkenyl group. The alkenyl group may have 2 to 8 carbon atoms. The alkenyl group for R^{A} may have terminal alkenyl functionality, e.g., R^{A} may have formula

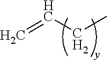

where subscript y is 0 to 6. Alternatively, each R^{A} may be independently selected from the group consisting of vinyl, allyl, and hexenyl. Alternatively, each R^{A} may be independently selected from the group consisting of vinyl and allyl. Alternatively, each R^{A} may be vinyl. Alternatively, each R^{A} may be allyl.

Alternatively, in formula (B1-1) when each R¹² is —OSi(R¹⁴)₃, each R¹⁴ may be —OSi(R¹⁵)₃ moieties such that the branched polyorganosiloxane oligomer has the following structure (B1-2):

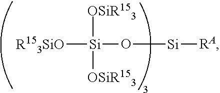

where R^{A} and R¹⁵ are as described above. Alternatively, each R may be an R¹³, as described above, and each R¹³ may be methyl.

Alternatively, in formula (B1-1), when each R¹² is —OSi(R¹⁴)₃, one R¹⁴ may be R¹³ in each —OSi(R¹⁴)₃ such that each R¹² is —OSiR¹³(R¹⁴)₂. Alternatively, two R¹⁴ in —OSiR¹³(R¹⁴)₂ may each be —OSi(R¹⁵)₃ moieties such that the branched polyorganosiloxane oligomer has the following structure (B1-3):

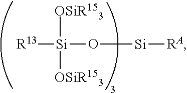

where R^{A}, R¹³, and R¹⁵ are as described above. Alternatively, each R¹⁵ may be an R¹³, and each R¹³ may be methyl.

Alternatively, in formula (B1-1), one R may be R¹³, and two of R¹² may be —OSi(R¹⁴)₃. When two of R¹² are —OSi(R¹⁴)₃, and one R¹⁴ is R¹³ in each —OSi(R¹⁴)₃ then two of R¹² are —OSiR¹³(R¹⁴)₂. Alternatively, each R¹⁴ in —OSiR¹³(R¹⁴)₂ may be —OSi(R¹⁵)₃ such that the branched polyorganosiloxane oligomer has the following structure (B1-4):

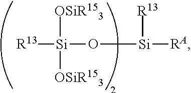

where R^{A}, R¹³, and R¹⁵ are as described above. Alternatively, each R¹⁵ may be an R¹³, and each R¹³ may be methyl. Alternatively, the alkenyl-functional branched polyorganosiloxane may have 3 to 16 silicon atoms per molecule, alternatively 4 to 16 silicon atoms per molecule, alternatively 4 to 10 silicon atoms per molecule, alternatively 7 to 16 silicon atoms per molecule, alternatively 7 to 10 silicon atoms per molecule, and alternatively 10 to 16 silicon atoms per molecule. Examples of alkenyl-functional branched polyorganosiloxane oligomers include vinyl-tris(trimethyl)siloxy)silane, which has formula (B1-5):

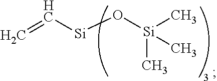

(1,1,1,3,5,7,9,9,9-nonamethyl-3,7-bis((trimethylsilyl)oxy)-5-vinylpentasiloxane), which has formula (B1-6):

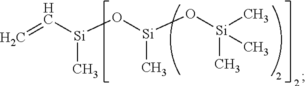

(5-((1,1,1,3,5,5,5-heptamethyltrisiloxan-3-yl)oxy)-,1,1,3,7,9,9,9-octamethyl-3,7-bis((trimethylsilyl)oxy)-5-vinylpentasiloxane, which has formula (B1-7)

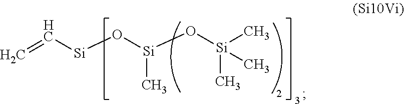

and
5-((1,1,1,3,5,5,5-heptamethyltrisiloxan-3-yl)oxy)-5-(hex-5-en-1-yl)-1,1,1,3,7,9,9,9-octamethyl-3,7-bis((trimethylsilyl)oxy)pentasiloxane, which has formula (B1-8):

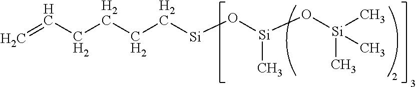

### (Si10-Hexenyl).

Branched alkenyl-functional polyorganosiloxane oligomers described above may be prepared by known methods, such as those disclosed in “Testing the Functional Tolerance of the Piers-Rubinsztajn Reaction: A new Strategy for Functional Silicones” by Grande, et al. Supplementary Material (ESI) for Chemical Communications, © The Royal Society of Chemistry 2010.

Alternatively, (B) the alkenyl-functional polyorganosiloxane may comprise (B2) a linear polydiorganosiloxane having, per molecule, at least one alkenyl group; alternatively at least two alkenyl groups. For example, said polydiorganosiloxane may comprise unit formula (B2-1): (R⁴₃SiO_{1/2})ₐ(R^{A}R⁴₂SiO_{1/2})_{b}(R⁴₂SiO_{2/2})_{c}(R^{A}R⁴SiO_{2/2})_{d}, where R^{A} and R⁴ are as described above, subscript a is 0, 1, or 2; subscript b is 0, 1, or 2, subscript c≥0, subscript d≥0, with the provisos that a quantity (b+d)≥1, a quantity (a+b)=2, and a quantity (a+b+c+d)≥2. Alternatively, in unit formula (B2-1) the quantity (a+b+c+d) may be at least 3, alternatively at least 4, and alternatively >50. At the same time in unit formula (B2-1), the quantity (a+b+c+d) may be less than or equal to 10,000; alternatively less than or equal to 4,000; alternatively less than or equal to 2,000; alternatively less than or equal to 1,000; alternatively less than or equal to 500; alternatively less than or equal to 250. Alternatively, in unit formula (B2-1) each R⁴ may be independently selected from the group consisting of alkyl and aryl; alternatively methyl and phenyl. Alternatively, each R⁴ in unit formula (B2-1) may be an alkyl group; alternatively each R⁴ may be methyl.

Alternatively, the polydiorganosiloxane of unit formula (B2-1) may have formula (B2-2):

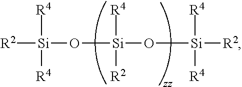

where R⁴ is as described above, R² is selected from the group consisting of R^{A} described above and R⁴, with the proviso that at least one R² per molecule is R^{A}, and subscript zz=a quantity (c+d). Alternatively, subscript zz may have a value of 0 to 2,000; alternatively 0 to 1,000; alternatively 50 to 2,000; alternatively 50 to 1,000; and alternatively 80 to 800.

Alternatively, this polydiorganosiloxane may comprise two different endgroups, i.e., when subscript a=1 and subscript b=1. Alternatively, this polydiorganosiloxane may be monofunctional, having one alkenyl group per molecule, e.g., when subscript a=1, b=1, and d=0 in unit formula (B2-1). This polydiorganosiloxane may have formula (B2-3):

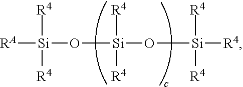

where R^{A} and R⁴ are as described above, and 9,998≥c≥0. Alternatively, in formula (B2-3), subscript c may have a value of 0 to 2,000; alternatively 0 to 1,000; alternatively 50 to 2,000; alternatively 50 to 1,000; and alternatively 80 to 800.

Alternatively, the polydiorganosiloxane of unit formula (B2-1) may be selected from the group consisting of: unit formula (B2-4): (R⁴₂R^{A}SiO_{1/2})₂(R⁴₂SiO_{2/2})ₘ(R⁴R^{A}SiO_{2/2})ₙ, unit formula (B2-5): (R⁴3SiO_{1/2})₂(R⁴₂SiO_{2/2})ₒ(R⁴R^{A}SiO_{2/2})ₚ, or a combination of both (B2-4) and (B2-5). n formulae (B2-4) and (B2-5), each R⁴ and R^{A} are as described above. Subscript m may be 0 or a positive number. Alternatively, subscript m may be at least 2. Alternatively subscript m be 2 to 2,000. Subscript n may be 0 or a positive number. Alternatively, subscript n may be 0 to 2000. Subscript o may be 0 or a positive number. Alternatively, subscript o may be 0 to 2000. Subscript p is at least 2. Alternatively subscript p may be 2 to 2000.

Alternatively, starting material (B2) may comprise an alkenyl-functional polydiorganosiloxane such as i) bis-dimethylvinylsiloxy-terminated polydimethylsiloxane, ii) bis-dimethylvinylsiloxy-terminated poly(dimethylsiloxane/methylvinylsiloxane), iii) bis-dimethylvinylsiloxy-terminated polymethylvinylsiloxane, iv) bis-trimethylsiloxy-terminated poly(dimethylsiloxane/methylvinylsiloxane), v) bis-trimethylsiloxy-terminated polymethylvinylsiloxane, vi) bis-dimethylvinylsiloxy-terminated poly(dimethylsiloxane/methylphenylsiloxane/methylvinylsiloxane), vii) bis-dimethylvinylsiloxy-terminated poly(dimethylsiloxane/methylphenylsiloxane), viii) bis-dimethylvinylsiloxy-terminated poly(dimethylsiloxane/diphenylsiloxane), ix) bis-phenyl,methyl,vinyl-siloxy-terminated polydimethylsiloxane, x) bis-dimethylhexenylsiloxy-terminated polydimethylsiloxane, xi) bis-dimethylhexenylsiloxy-terminated poly(dimethylsiloxane/methylhexenylsiloxane), xii) bis-dimethylhexenylsiloxy-terminated polymethylhexenylsiloxane, xiii) bis-trimethylsiloxy-terminated poly(dimethylsiloxane/methylhexenylsiloxane), xiv) bis-trimethylsiloxy-terminated polymethylhexenylsiloxane, xv) bis-dimethylhexenyl-siloxy terminated poly(dimethylsiloxane/methylphenylsiloxane/methylhexenylsiloxane), xvi) bis-dimethylvinylsiloxy-terminated poly(dimethylsiloxane/methylhexenylsiloxane), xvii) bis-dimethylhexenyl-siloxy-terminated poly(dimethylsiloxane/methylphenylsiloxane), xviii) bis-dimethylhexenyl-siloxy-terminated poly(dimethylsiloxane/diphenylsiloxane), xix) alpha-dimethyl(n-butyl)siloxy-omega-dimethylvinylsiloxy-terminated poly(dimethylsiloxane), and xx) a combination of two or more of i) to xix).

Methods of preparing linear alkenyl-functional polydiorganosiloxanes described above for starting material (B2), such as hydrolysis and condensation of the corresponding organohalosilanes and oligomers or equilibration of cyclic polydiorganosiloxanes, are known in the art, see for example U.S. Pat. Nos. 3,284,406; 4,772,515; 5,169,920; 5,317,072; and 6,956,087, which disclose preparing linear polydiorganosiloxanes with alkenyl groups. Examples of linear polydiorganosiloxanes having alkenyl groups are commercially available from, e.g., Gelest Inc. of Morrisville, Pennsylvania, USA under the tradenames DMS-V00, DMS-V03, DMS-V05, DMS-V21, DMS-V22, DMS-V25, DMS-V-31, DMS-V33, DMS-V34, DMS-V35, DMS-V41, DMS-V42, DMS-V43, DMS-V46, DMS-V51, DMS-V52, MCR-V21, MCR-V25, and MCR-41.

### (C) Hydroformylation Reaction Catalyst

Starting material (C), the hydroformylation reaction catalyst, comprises an activated complex of rhodium and a close ended bisphosphite ligand. The bisphosphite ligand may be symmetric or asymmetric. Alternatively, the bisphosphite ligand may be symmetric. The bisphosphite ligand may have formula (C1):

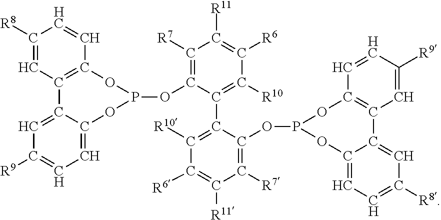

where R⁶ and R^{6′} are each independently selected from the group consisting of hydrogen, an alkyl group of at least one carbon atom, a cyano group, a halogen group, and an alkoxy group of at least one carbon atom; R⁷ and R^{7′} are each independently selected from the group consisting of an alkyl group of at least 3 carbon atoms and a group of formula —SiR¹⁷₃, where each R¹⁷ is an independently selected monovalent hydrocarbon group of 1 to 20 carbon atoms; R⁸, R^{8′}, R⁹, and R^{9′} are each independently selected from the group consisting of hydrogen, an alkyl group, a cyano group, a halogen group, and an alkoxy group; and R¹⁰, R^{10′}, R¹¹, and R^{11′} are each independently selected from the group consisting of hydrogen and an alkyl group. Alternatively, one of R⁷ and R^{7′} may be hydrogen.

In formula (C1), R⁶ and R^{6′} may be alkyl groups of least one carbon atom, alternatively 1 to 20 carbon atoms. Suitable alkyl groups for R⁶ and R^{6′} may be linear, branched, cyclic, or combinations of two or more thereof. The alkyl groups are exemplified by methyl, ethyl, propyl (including n-propyl and/or isopropyl), butyl (including n-butyl, tert-butyl, sec-butyl, and/or isobutyl); pentyl, hexyl, heptyl, octyl, decyl, dodecyl, undecyl, and octadecyl (and branched isomers having 5 to 20 carbon atoms), and the alkyl groups are further exemplified by cycloalkyl groups such as cyclopropyl, cyclobutyl, cyclopentyl, and cyclohexyl. Alternatively, the alkyl group for R⁶ and R^{6′} may be selected from the group consisting of ethyl, propyl and butyl; alternatively propyl and butyl. Alternatively, the alkyl group for R⁶ and R^{6′} may be butyl. Alternatively, R⁶ and R^{6′} may be alkoxy groups, wherein the alkoxy group may have formula —OR^{6″}, where R^{6″} is an alkyl group as described above for R⁶ and R^{6′}.

Alternatively, in formula (C1), R⁶ and R^{6′} may be independently selected from alkyl groups of 1 to 6 carbon atoms and alkoxy groups of 1 to 6 carbon atoms. Alternatively, R⁶ and R^{6′} may be alkyl groups of 2 to 4 carbon atoms. Alternatively, R⁶ and R^{6′} may be alkoxy groups of 1 to 4 carbon atoms. Alternatively, R⁶ and R^{6′} may be butyl groups, alternatively tert-butyl groups. Alternatively, R⁶ and R^{6′} may be methoxy groups.

In formula (C1), R⁷ and R^{7′} may be alkyl groups of least three carbon atoms, alternatively 3 to 20 carbon atoms. Suitable alkyl groups for R⁷ and R^{7′} may be linear, branched, cyclic, or combinations of two or more thereof. The alkyl groups are exemplified by propyl (including n-propyl and/or isopropyl), butyl (including n-butyl, tert-butyl, sec-butyl, and/or isobutyl); pentyl, hexyl, heptyl, octyl, decyl, dodecyl, undecyl, and octadecyl (and branched isomers having 5 to 20 carbon atoms), and the alkyl groups are further exemplified by cycloalkyl groups such as cyclopropyl, cyclobutyl, cyclopentyl, and cyclohexyl. Alternatively, the alkyl group for R⁷ and R^{7′} may be selected from the group consisting of propyl and butyl. Alternatively, the alkyl group for R⁷ and R^{7′} may be butyl.

Alternatively, in formula (C1), R⁷ and R^{7′} may be a silyl group of formula —SiR¹⁷₃, where each R¹⁷ is an independently selected monovalent hydrocarbon group of 1 to 20 carbon atoms. The monovalent hydrocarbon group may be an alkyl group of 1 to 20 carbon atoms, as described above for R⁶ and R^{6′}.

Alternatively, in formula (C1), R⁷ and R^{7′} may each be independently selected alkyl groups, alternatively alkyl groups of 3 to 6 carbon atoms. Alternatively, R⁷ and R^{7′} may be alkyl groups of 3 to 4 carbon atoms. Alternatively, R⁷ and R^{7′} may be butyl groups, alternatively tert-butyl groups.

In formula (C1), R⁸, R^{8′}, R⁹, R^{9′} may be alkyl groups of at least one carbon atom, as described above for R⁶ and R^{6′}. Alternatively, R⁸ and R^{8′} may be independently selected from the group consisting of hydrogen and alkyl groups of 1 to 6 carbon atoms. Alternatively, R⁸ and R^{8′} may be hydrogen. Alternatively, in formula (C1), R^{9′} and R^{9′} may be independently selected from the group consisting of hydrogen and alkyl groups of 1 to 6 carbon atoms. Alternatively, R⁹ and R^{9′} may be hydrogen.

In formula (C1), R¹⁰ and R^{10′} may be hydrogen atoms or alkyl groups of least one carbon atom, alternatively 1 to 20 carbon atoms. The alkyl groups for R¹⁰ and R^{10′} may be as described above for R⁶ and R^{6′}. Alternatively, R¹⁰ and R^{10′} may be methyl. Alternatively, R¹⁰ and R^{10′} may be hydrogen.

In formula (C1), R¹¹ and R^{11′} may be hydrogen atoms or alkyl groups of least one carbon atom, alternatively 1 to 20 carbon atoms. The alkyl groups for R¹ and R^{11′} may be as described above for R⁶ and R^{6′}. Alternatively, R¹¹ and R^{11′} may be hydrogen.

Alternatively, the ligand of formula (C1) may be selected from the group consisting of (C1-1) 6,6′-[[3,3′,5,5′-tetrakis(1,1-dimethylethyl)-1,1′-biphenyl]-2,2′-diyl]bis(oxy)]bis-dibenzo[d,f][1,3,2]dioxaphosphepin; (C1-2) 6,6′-[(3,3′-di-tert-butyl-5,5′-dimethoxy-1,1′-biphenyl-2,2′-diyl)bis(oxy)]bis(dibenzo[dj][1,3,2]dioxaphosphepin); and a combination of both (C1-1) and (C1-2).

Alternatively, the ligand may comprise 6,6′-[[3,3′,5,5′-tetrakis(1,1-dimethylethyl)-1,1′-biphenyl]-2,2′-diyl]bis(oxy)]bis-dibenzo[d,f][1,3,2]dioxaphosphepin, as disclosed at col. 11 of U.S. Pat. No. 10,023,516 (see also U.S. Pat. No. 7,446,231, which discloses this compound as Ligand D at col. 22 and U.S. Pat. No. 5,727,893 at col. 20, lines 40-60 as ligand F).

Alternatively, the ligand may comprise biphephos, which is commercially available from Sigma Aldrich and may be prepared as described in U.S. Pat. No. 9,127,030. (See also U.S. Pat. No. 7,446,231 ligand B at col. 21 and U.S. Pat. No. 5,727,893 at col. 20, lines 5-18 as ligand D).

Starting material (C), the rhodium/bisphosphite ligand complex catalyst, may be prepared by methods known in the art, such as those disclosed in U.S. Pat. No. 4,769,498 to Billig, et al. at col. 20, line 50-col. 21, line 40 and U.S. Pat. No. 10,023,516 to Brammer et al. col. 11, line 35-col. 12, line 12 by varying appropriate starting materials. For example, the rhodium/bisphosphite ligand complex may be prepared by a process comprising combining a rhodium precursor and the bisphosphite ligand (C1) described above under conditions to form the complex, which complex may then be introduced into a hydroformylation reaction medium comprising one or both of starting materials (A) and/or (B), described above. Alternatively, the rhodium/bisphosphite ligand complex may be formed in situ by introducing the rhodium catalyst precursor into the reaction medium, and introducing (C1) the bisphosphite ligand into the reaction medium (e.g., before, during, and/or after introduction of the rhodium catalyst precursor), for the in situ formation of the rhodium/bisphosphite ligand complex. The rhodium/bisphosphite ligand complex can be activated by heating and/or exposure to starting material (A) to form the (C) rhodium/bisphosphite ligand complex catalyst. Rhodium catalyst precursors are exemplified by rhodium dicarbonyl acetylacetonate, Rh₂O₃, Rh₄(CO)₁₂, Rh₆(CO)₁₆, and Rh(NO₃)₃.

For example, a rhodium precursor, such as rhodium dicarbonyl acetylacetonate, optionally starting material (D), a solvent, and (C1) the bisphosphite ligand may be combined, e.g., by any convenient means such as mixing. The resulting rhodium/bisphosphite ligand complex may be introduced into the reactor, optionally with excess bisphosphite ligand. Alternatively, the rhodium precursor, (D) the solvent, and the bisphosphite ligand may be combined in the reactor with starting material (A) and/or (B), the alkenyl-functional polyorganosiloxane; and the rhodium/bisphosphite ligand complex may form in situ. The relative amounts of bisphosphite ligand and rhodium precursor are sufficient to provide a molar ratio of bisphosphite ligand/Rh of 10/1 to 1/1, alternatively 5/1 to 1/1, alternatively 3/1 to 1/1, alternatively 2.5/1 to 1.5/1. In addition to the rhodium/bisphosphite ligand complex, excess (e.g., not complexed) bisphosphite ligand may be present in the reaction mixture. The excess bisphosphite ligand may be the same as, or different from, the bisphosphite ligand in the complex.

The amount of (C) the rhodium/bisphosphite ligand complex catalyst (catalyst) is sufficient to catalyze hydroformylation of (B) the alkenyl-functional polyorganosiloxane. The exact amount of catalyst will depend on various factors including the type of alkenyl-functional polyorganosiloxane selected for starting material (B), its exact alkenyl content, and the reaction conditions such as temperature and pressure of starting material (A). However, the amount of (C) the catalyst may be sufficient to provide a rhodium metal concentration of at least 0.1 ppm, alternatively 0.15 ppm, alternatively 0.2 ppm, alternatively 0.25 ppm, and alternatively 0.5 ppm, based on the weight of (B) the alkenyl-functional polyorganosiloxane. At the same time, the amount of (C) the catalyst may be sufficient to provide a rhodium metal concentration of up to 300 ppm, alternatively up to 100 ppm, alternatively up to 20 ppm, and alternatively up to 5 ppm, on the same basis. Alternatively, the amount of (C) the catalyst may be sufficient to provide 0.1 ppm to 300 ppm, alternatively 0.2 ppm to 100 ppm, alternatively, 0.25 ppm to 20 ppm, and alternatively 0.5 ppm to 5 ppm, based on the weight of (B) the alkenyl-functional polyorganosiloxane.

### (D) Solvent (Suitable for Use in Hydroformylation Reaction)

The hydroformylation reaction may run without additional solvents. Alternatively, the hydroformylation reaction may be carried out with (D) a solvent, which is suitable for use in a hydroformylation reaction, for example to facilitate mixing and/or delivery of one or more of the starting materials described above, such as the (C) catalyst and/or starting material (B), when a solvent such as an alkenyl-functional polyorganosilicate resin is selected for starting material (B). The solvent is exemplified by aliphatic or aromatic hydrocarbons, which can dissolve the starting materials, e.g., toluene, xylene, benzene, hexane, heptane, decane, cyclohexane, or a combination of two or more thereof. Additional solvents include tetrahydrofuran (THF), dibutyl ether, diglyme, and Texanol. Without wishing to be bound by theory, it is thought that solvent may be used to reduce the viscosity of the starting materials. The amount of solvent is not critical, however, when present, the amount of solvent may be 5% to 70% based on weight of starting material (B) the alkenyl-functional polyorganosiloxane.

### Hydrogenation

The process for making the macromonomer described herein may further comprise preparing (I) the carboxy-functional polyorganosiloxane by a process comprising: 3) combining, under conditions to conduct oxidation reaction, starting materials comprising (E) the aldehyde-functional polyorganosiloxane described above, (F) an oxygen source, optionally (G) an oxidation reaction catalyst, and optionally (H) a (second) solvent (which is suitable for use in oxidation reaction); thereby forming an oxidation reaction product comprising (I) the carboxy-functional polyorganosiloxane. Alternatively, in addition to the steps recited above, the process may optionally further comprise: drying one or more of starting materials (E), (F), (G), and (H) before oxidation reaction, e.g., in step 3). The process may optionally further comprise: 4) recovering the carboxy-functional polyorganosiloxane from the oxidation reaction product. Step 4) may be performed during and/or after step 3).

### (E) Aldehyde-Functional Polyorganosiloxane

Starting material (E) is the aldehyde-functional polyorganosiloxane, which has, per molecule, at least one aldehyde-functional group covalently bonded to silicon. Alternatively, the aldehyde-functional organosilicon compound may have, per molecule, more than one aldehyde-functional group covalently bonded to silicon. The aldehyde-functional group covalently bonded to silicon may have formula:

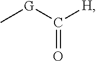

where G is a divalent hydrocarbon group free of aliphatic unsaturation that has 2 to 8 carbon atoms, as described above.

The aldehyde-functional polyorganosiloxane may be branched., e.g., a branched oligomer. This branched oligomer may have general formula (E1-1): R^{Ald}SiR¹²₃, where R^{Ald} has formula

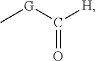

where G is as described above, and each R¹² is selected from R¹³ and —OSi(R¹⁴)₃; where each R¹³ is a monovalent hydrocarbon group; where each R¹⁴ is selected from R¹³, —OSi(R¹⁵)₃, and —[OSiR¹³₂]ᵢᵢOSiR¹³₃; where each R¹⁵ is selected from R¹³, —OSi(R¹⁶)₃, and —[OSiR¹³₂]ᵢᵢOSiR¹³₃; where each R¹⁶ is selected from R¹³ and —[OSiR¹³₂]ᵢᵢOSiR¹³₃; and where subscript ii has a value such that 0≤ii≤100. At least two of R¹² may be —OSi(R¹⁴)₃. Alternatively, all three of R¹² may be —OSi(R¹⁴)₃.

Alternatively, in formula (E1-1) when each R¹² is —OSi(R¹⁴)₃, each R¹⁴ may be —OSi(R¹⁵)₃ moieties such that the branched polyorganosiloxane oligomer has the following structure (E1-2):

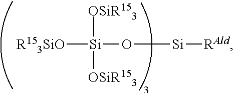

where R^{Ald} and R¹⁵ are as described above. Alternatively, each R¹⁵ may be an R³, as described above, and each R¹³ may be methyl.

Alternatively, in formula (E1-1), when each R¹² is —OSi(R¹⁴)₃, one R¹⁴ may be R¹³ in each —OSi(R¹⁴)₃ such that each R¹² is —OSiR¹³(R¹⁴)₂. Alternatively, two R¹⁴ in —OSiR¹³(R¹⁴)₂ may each be —OSi(R¹⁵)₃ moieties such that the branched aldehyde-functional polyorganosiloxane oligomer has the following structure (E1-3):

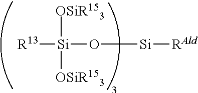

where R^{Ald}, R¹³, and R¹⁵ are as described above. Alternatively, each R¹⁵ may be an R¹³, and each R¹³ may be methyl.

Alternatively, in formula (E1-1), one R² may be R¹³, and two of R¹² may be —OSi(R¹⁴)₃. When two of R¹² are —OSi(R¹⁴)₃, and one R¹⁴ is R¹³ in each —OSi(R¹⁴)₃ then two of R¹² are —OSiR¹³(R¹⁴)₂. Alternatively, each R¹⁴ in —OSiR¹³(R¹⁴)₂ may be —OSi(R¹⁵)₃ such that the branched polyorganosiloxane oligomer has the following structure (E1-4):

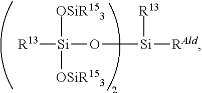

where R^{Ald}, R¹³, and R¹⁵ are as described above. Alternatively, each R¹⁵ may be an R¹³, and each R¹³ may be methyl. Alternatively, the aldehyde-functional branched polyorganosiloxane may have 3 to 16 silicon atoms per molecule, alternatively 4 to 16 silicon atoms per molecule, alternatively 4 to 10 silicon atoms per molecule, alternatively 7 to 16 silicon atoms per molecule, alternatively 7 to 10 silicon atoms per molecule, and alternatively 10 to 16 silicon atoms per molecule. Examples of aldehyde-functional branched polyorganosiloxane oligomers include 3-(1,1,1,5,5,5-hexamethyl-3-((trimethylsilyl)oxy)trisiloxan-3-yl)propanal (which can also be named propyl-aldehyde-tris(trimethylsiloxy)silane), which has formula (E1-5):

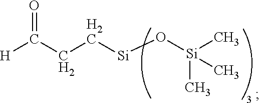

3-(1,1,1,3,5,7,9,9,9-nonamethyl-3,7-bis((trimethylsilyl)oxy)pentasiloxan-5-yl)propanal (which can also be named methyl-(propyl-aldehyde)-di((1,1,1,3,5,5,5-heptamethyltrisiloxan-3-yl)oxy)-silane), which has formula (E1-6):

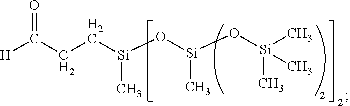

3-(5-((1,1,1,3,5,5,5-heptamethyltrisiloxan-3-yl)oxy)-1,1,1,3,7,9,9,9-octamethyl-3,7-bis((trimethylsilyl)oxy)pentasiloxan-5-yl)propanal (which can also be named (propyl-aldehyde)-tris((1,1,1,3,5,5,5-heptamethyltrisiloxan-3-yl)oxy)-silane), which has formula (Et-7):

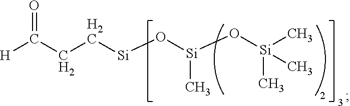

and
7-(5-((1,1,1,3,5,5,5-heptamethyltrisiloxan-3-yl)oxy)-1,1,1,3,7,9,9,9-octamethyl-3,7-bis((trimethylsilyl)oxy)pentasiloxan-5-yl)heptanal (which can also be named (heptyl-aldehyde)-tris((1,1,1,3,5,5,5-heptamethyltrisiloxan-3-yl)oxy)-silane), which has formula (E1-8):

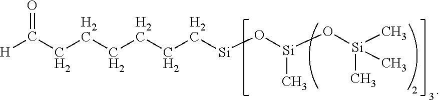

Alternatively, (E) the aldehyde-functional polyorganosiloxane may comprise (E2) a linear polydiorganosiloxane having, per molecule, at least one aldehyde-functional group; alternatively at least two aldehyde-functional groups. For example, said polydiorganosiloxane may comprise unit formula (E2-1): (R⁴₃SiO_{1/2})ₐ(R^{Ald}R⁴₂SiO_{1/2})_{b}(R⁴₂SiO_{2/2})_{c}(R^{Ald}R⁴SiO_{2/2})_{d}, where R^{Ald} is as described above for formula (E1-1), R⁴ is as described above for formula (B1-1) and subscripts a, b, c, and d are as described above for unit formula (B2-1).

Alternatively, the polydiorganosiloxane of unit formula (E2-1) may have formula (E2-2):

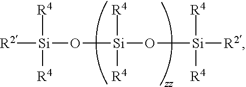

where each R^{2′} is independently selected from the group consisting of R⁴ and R^{Ald}, with the proviso that at least one R_{2′} per molecule is R^{Ald}, each R^{Ald} is as described above for formula (E1-1), each R⁴ is as described above for formula (B1-1), and subscript zz is as described above for formula (B2-2).

Alternatively, this polydiorganosiloxane may comprise two different endgroups, i.e., when subscript a=1 and subscript b=1. Alternatively, this polydiorganosiloxane may be monofunctional, having one aldehyde-functional group per molecule, e.g., when subscript a=1, b=1, and d=0 in unit formula (E2-1). This polydiorganosiloxane may have formula (E2-3):

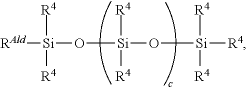

where R^{Ald} is as described above for formula (E1-1), each R⁴ is as described above for formula (B1-1), and subscript c is as described above for formula (B2-3).

Alternatively, the linear aldehyde-functional polydiorganosiloxane of unit formula (E2-1) may be selected from the group consisting of: unit formula (E2-4): (R⁴₂R^{Ald}SiO_{1/2})₂(R⁴R^{Ald}SiO_{2/2})ₙ, unit formula (E2-5): (R⁴₃SiO_{1/2})₂(R⁴₂SiO_{2/2})ₒ(R⁴R^{Ald}SiO_{2/2})ₚ, or a combination of both (E2-4) and (E2-5). In formulae (E2-4) and (E2-5), each R^{Ald} is as described above for formula (E1-1), each R⁴ is as described above for formula (B1-1), and subscripts m, n, o, and p are as described above for formulas (B2-4) and (B2-5).

Starting material (E2) may comprise an aldehyde-functional polydiorganosiloxane such as i) bis-dimethyl(propyl-aldehyde)siloxy-terminated polydimethylsiloxane, ii) bis-dimethyl(propyl-aldehyde)siloxy-terminated poly(dimethylsiloxane/methyl(propyl-aldehyde)siloxane), iii) bis-dimethyl(propyl-aldehyde)siloxy-terminated polymethyl(propyl-aldehyde)siloxane, iv) bis-trimethylsiloxy-terminated poly(dimethylsiloxane/methyl(propyl-aldehyde)siloxane), v) bis-trimethylsiloxy-terminated polymethyl(propyl-aldehyde)siloxane, vi) bis-dimethyl(propyl-aldehyde)siloxy-terminated poly(dimethylsiloxane/methylphenylsiloxane/methyl(propyl-aldehyde)siloxane), vii) bis-dimethyl(propyl-aldehyde)siloxy-terminated poly(dimethylsiloxane/methylphenylsiloxane), viii) bis-dimethyl(propyl-aldehyde)siloxy-terminated poly(dimethylsiloxane/diphenylsiloxane), ix) bis-phenyl,methyl,(propyl-aldehyde)-siloxy-terminated polydimethylsiloxane, x) bis-dimethyl(heptyl-aldehyde)siloxy-terminated polydimethylsiloxane, xi) bis-dimethyl(heptyl-aldehyde)siloxy-terminated poly(dimethylsiloxane/methyl(heptyl-aldehyde)siloxane), xii) bis-dimethyl(heptyl-aldehyde)siloxy-terminated polymethyl(heptyl-aldehyde)siloxane, xiii) bis-trimethylsiloxy-terminated poly(dimethylsiloxane/methyl(heptyl-aldehyde)siloxane), xiv) bis-trimethylsiloxy-terminated polymethyl(heptyl-aldehyde)siloxane, xv) bis-dimethyl(heptyl-aldehyde)-siloxy terminated poly(dimethylsiloxane/methylphenylsiloxane/methyl(heptyl-aldehyde)siloxane), xvi) bis-dimethyl(propyl-aldehyde)siloxy-terminated poly(dimethylsiloxane/methyl(heptyl-aldehyde)siloxane), xvii) bis-dimethyl(heptyl-aldehyde)-siloxy-terminated poly(dimethylsiloxane/methylphenylsiloxane), xviii) bis-dimethyl(heptyl-aldehyde)-siloxy-terminated poly(dimethylsiloxane/diphenylsiloxane), xix) alpha-dimethyl(n-butyl)siloxy-omega-dimethyl(propyl-aldehyde)siloxy-terminated poly(dimethylsiloxane), and xx) a combination of two or more of i) to xix).

### (F) Oxygen Source

Oxygen sources are known in the art and readily available. For example, the oxygen source may be ambient air, which comprises 21% oxygen. Alternatively, the oxygen source may be concentrated or purified oxygen, e.g., any gas stream with 21% to 100% oxygen. Pure or nearly pure (>99% pure) oxygen is known in the art and commercially available from various sources, e.g., Air Products of Allentown, Pennsylvania, USA. Alternatively, the oxygen source may comprise a peroxide compound (i.e., a compound having at least one —O—O— group per molecule). Suitable peroxide compounds include an organic hydroperoxide such as an alkyl hydroperoxide (e.g., tert-butyl hydroperoxide), a dialkyl peroxide (e.g., di-tert-butyl peroxide), a peroxyacid (such as 3-chloroperbenzoic acid), or a combination thereof. The oxygen source may be used in an amount sufficient to provide a superstoichiometric amount of oxygen with respect to the aldehyde-functionality of starting material (E), the aldehyde-functional polyorganosiloxane described above. The amount of oxygen source (and reaction conditions) is sufficient to permit oxidation of at least one of the aldehyde-functional groups, per molecule, of the aldehyde-functional polyorganosiloxane. Alternatively, some of the aldehyde-functional groups may be converted to carboxylic acid groups. Alternatively, complete conversion of aldehyde-functional groups to carboxylic acid functional groups may be performed.

### (G) Oxidation Reaction Catalyst

The oxidation reaction catalyst used in the process (in step 3)) for preparing the carboxy-functional polyorganosiloxane may be a heterogeneous oxidation reaction catalyst, a homogenous oxidation reaction catalyst, or a combination thereof.

An exemplary oxidation reaction catalyst may comprise a metal complex or compound. The metal complex or compound may comprise a metal selected from the group consisting of cobalt (Co), copper (Cu), iron (Fe), Manganese (Mn), Nickel (Ni), Rhodium (Rh), Selenium (Se), and Tungsten (W), and combinations of two or more thereof. For example, manganese acetate (Mn(OAc)₂) may be used. Non-metal based catalysts may also be suitable, such as those described in RSC Adv., 2013, 3, 18931-18937. The metal complex may further comprise a ligand, such as acetate. Alternatively, the oxidation reaction catalyst may comprise Rh. Without wishing to be bound by theory, it is thought that when the hydroformylation process described above is used to make the aldehyde-functional polyorganosiloxane, and the Rh complex used as (C) the hydroformylation reaction catalyst is present, this Rh complex may serve as oxidation reaction catalyst. Alternatively, (G) the oxidation reaction catalyst may comprise an organocatalyst containing N-hydroxy functionality. Exemplary organocatalysts include N-hydroxyphthalimide or 2,2,6,6-tetramethylpiperidin-1-yl)oxyl (TEMPO). Suitable oxidation reaction catalysts are known in the art and are commercially available. For example, N-hydroxyphthalimide and TEMPO are commercially available from various sources including Sigma-Aldrich, Inc. of St. Louis, Missouri, USA.

The amount of (G) the oxidation reaction catalyst used in step 3) of the process depends on various factors including whether the process will be run in a batch or continuous mode, the selection of aldehyde-functional polyorganosiloxane, whether a heterogeneous or homogeneous oxidation reaction catalyst is selected, and reaction conditions such as temperature and pressure. However, the amount of catalyst (for the batch process or a continuous process using a homogeneous oxidation catalyst) may be 0.001 mole % to 1 mole %, alternatively 0.005 mole % to 0.5 mole %, based on moles of the aldehyde-functional group in starting material (E) the aldehyde-functional polyorganosiloxane. Alternatively, the amount of catalyst may be at least 0.001, alternatively at least 0.005, alternatively at least 0.01, and alternatively at least 0.1, mole %; while at the same time the amount of catalyst may be up to 1, alternatively up to 0.75, alternatively up to 0.5, alternatively up to 0.25, and alternatively up to 0.1, mole %, on the same basis. Alternatively, when the oxidation reaction will be run in a continuous mode, e.g., by packing a fixed bed reactor with a heterogeneous oxidation reaction catalyst, the amount of the oxidation reaction catalyst may be sufficient to provide a reactor volume (filled with oxidation reaction catalyst) to achieve a space time of 10 hr⁻¹, or catalyst surface area sufficient to achieve 8 to 10 kg/hr substrate per m² of catalyst.

(H) Second Solvent (suitable for use in oxidation reaction process)

A solvent that may optionally be used in step 3) of the process, to facilitate the oxidation reaction, may be selected from those solvents that are neutral to the oxidation reaction. The following are specific examples of such solvents: ketones such as acetone and 3-pentanone; esters; carboxylic acids; aliphatic hydrocarbons, such as hexane, heptane, and paraffinic solvents; and aromatic hydrocarbons such as benzene, toluene, ethylbenzene, and xylene. These solvents can be used individually or in combinations of two or more. This second solvent is starting material (H), and may be the same as, or different from, starting material (D) when the hydroformylation process described above is used to prepare the aldehyde-functional polyorganosiloxane used as starting material (E).

### Oxidation Reaction Conditions

The oxidation reaction in step 3) can be performed using a pressurized oxygen source.

Partial pressure of the oxygen may be 3 psia (20 kPa) to 100 psia (690 kPa), alternatively 3 psia (20 kPa) to 15 psia (104 kPa). The reaction may be carried out at a temperature of 0 to 200° C. The temperature in step 3) may depend on various factors such as the pressure selected, the aldehyde-functional polyorganosiloxane selected, and the reactor configuration. Without wishing to be bound by theory, it is thought that oxidation reaction rate may increase as temperature increases, but oxygen solubility in the aldehyde-functional polyorganosiloxane may decreases as temperature increase, therefore, temperature may be selected so as to have sufficient oxygen solubility to allow the oxidation reaction to proceed while maximizing reaction rate. The temperature may be, for example, 0° C. to 100° C. Alternatively, a temperature of 23° C. to 100° C., and alternatively 20° C. to 50° C., may be suitable. Alternatively, the oxygen source partial pressure used may be at least 3, alternatively at least 4, alternatively at least 6, alternatively at least 8, and alternatively at least 10, psia; while at the same time the pressure may be up to 100, alternatively up to 75, alternatively up to 50, alternatively up to 25, and alternatively up to 15, psia. The temperature for oxidation reaction may be at least 20, alternatively at least 25, alternatively at least 30, ° C., while at the same time the temperature may be up to 100, alternatively up to 95, and alternatively up to 90, ° C.

The oxidation reaction can be carried out in a batch or a continuous mode. In a batch mode, the reaction time depends on various factors including the amount of the catalyst and reaction temperatures, however, step 3) of the process described herein may be performed for 1 minute to 250 hours. Alternatively, the oxidation reaction may be performed for at least 1 minute, alternatively at least 2 minutes, alternatively at least 1 hour, alternatively at least 2.5 hours, alternatively at least 3 hours, alternatively at least 3.3 hours, alternatively at least 3.7 hours, alternatively at least 4 hours, alternatively at least 4.4 hours, and alternatively at least 5.5 hours; while at the same time, the oxidation reaction may be performed for up to 250 hours, alternatively 200 hours, alternatively up to 175 hours, alternatively up to 150 hours, alternatively up to 125 hours, alternatively up to 100 hours, and alternatively up to 75 hours.

Alternatively, in a batch mode, the terminal point of an oxidation reaction can be considered to be the time during which the decrease in pressure of the oxygen source is no longer observed after the reaction is continued for an additional 1 to 2 hours. If oxygen source pressure decreases in the course of the reaction, it may be desirable to repeat the introduction of the oxygen source and to maintain it under increased pressure to shorten the reaction time. Alternatively, the reactor can be re-pressurized with the oxygen source 1 or more times to achieve sufficient supply of oxygen for reaction of the aldehyde while maintaining reasonable reactor pressures. The same oxygen source, or a different oxygen source (e.g., more concentrated in O₂) may be used when re-pressurizing the reactor to finish oxidation of the aldehyde.

The oxidation reaction in step 3) may optionally further comprise irradiating the reaction mixture with ultra-violet (UV) radiation. UV radiation may have a peak wavelength of 285 nm and may be provided by any convenient means such as an LED or other lamp. Alternatively, UV radiation with a wavelength of 200 nm to 460 nm; alternatively 250 nm to 350 nm, and alternatively 265 nm to 315 nm may be used. The exposure dose depends on various factors including the wavelength selected and other reaction conditions. For example, a UV dosage of 9 μW/cm² may be used. Without wishing to be bound by theory, it is thought that UV irradiation may increase rate of the oxidation reaction in step 3).

After the oxidation reaction, the oxidation reaction catalyst may be separated in a pressurized atmosphere by any convenient means, such as filtration or adsorption, e.g., with diatomaceous earth or activated carbon, settling, centrifugation, by maintaining the oxidation reaction catalyst in a structured packing or other fixed structure, or a combination thereof (e.g., in step 4)).

The carboxy-functional polyorganosiloxane prepared as described above has, per molecule, at least one carboxy-functional group covalently bonded to silicon. Alternatively, the carboxy-functional polyorganosiloxane may have, per molecule, more than one carboxy-functional group covalently bonded to silicon. The carboxy-functional group covalently bonded to silicon, R^{Car}, may have formula:

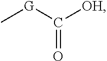

where G is a divalent hydrocarbon group free of aliphatic unsaturation that has 2 to 8 carbon atoms, as described and exemplified above. Alternatively, each R^{Car} may be independently selected from the group consisting of —(C₂H₄)C(═O)OH, —(C₃H₆)C(═O)OH, and —(C₆H₁₂)C(═O)OH. The carboxy-functional polyorganosiloxane may have any one of the formulas shown above for (E) the aldehyde-functional polyorganosiloxane, with the proviso that one or more instances of R^{Ald} is replaced with R^{Car}.

### Transvinylation

The process for preparing the vinylester-functional siloxane macromonomer described herein comprises: combining, under conditions to conduct transvinylation reaction, starting materials comprising
(I) the carboxy-functional polyorganosiloxane described above,
(J) a vinyl acetate-functional compound of formula

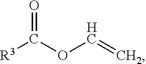

where R³ is an alkyl group of 1 to 6 carbon atoms,
(K) a transvinylation reaction catalyst,
optionally (L) a (third) solvent, and
optionally (X) an inhibitor;
   thereby preparing a reaction mixture comprising the vinyl-ester functional macromonomer.

The transvinylation reaction may be performed in step 5), when the process including hydroformylation reaction and oxidation reaction, as described above, is used to prepare (I) the carboxy-functional polyorganosiloxane.

The transvinylation reaction (e.g., in step 5)) can be performed at ambient pressure. The transvinylation reaction may be carried out at a temperature of 0 to 150° C. The temperature for transvinylation reaction may depend on various factors, including the carboxy-functional organosilicon compound selected and the reactor configuration. The temperature may be, for example, 0° C. to 150° C. Alternatively, a temperature of 23° C. to 100° C., alternatively 30° C. to 80° C., alternatively 40° C. to 70° C., and alternatively 50° C. to 60° C. may be suitable.

The transvinylation reaction can be carried out in a batch or a continuous mode. In a batch mode, the reaction time depends on various factors including the amount of the transvinylation reaction catalyst and reaction temperatures, however, transvinylation reaction may be performed for 1 minute to 250 hours. Alternatively, the oxidation reaction may be performed for at least 1 minute, alternatively at least 2 minutes, alternatively at least 1 hour, alternatively at least 2.5 hours, alternatively at least 3 hours, alternatively at least 3.3 hours, alternatively at least 3.7 hours, alternatively at least 4 hours, alternatively at least 4.4 hours, and alternatively at least 5.5 hours; while at the same time, the transvinylation reaction may be performed for up to 250 hours, alternatively 200 hours, alternatively up to 175 hours, alternatively up to 150 hours, alternatively up to 125 hours, alternatively up to 100 hours, and alternatively up to 75 hours.

The process for preparing the vinylester-functional siloxane macromonomer may optionally further comprise recovering the vinyl-ester functional macromonomer. This may be performed in step 6), when the process including hydroformylation reaction and oxidation reaction is used to prepare the carboxy-functional polyorganosiloxane described above. Recovering the macromonomer may be performed by any convenient means such as filtration, stripping, and/or distillation, optionally with heating and/or reduced pressure.

### (I) Carboxy-functional Polyorganosiloxane

Starting material (I) in the process for preparing the vinyl-ester functional macromonomer is a carboxy-functional polyorganosiloxane. Commercially available carboxy-functional organosilicon compounds may be used in addition to, or instead of, the carboxy-functional polyorganosiloxane prepared by the process including hydroformylation reaction and oxidation reaction described above. For example, MCR-B12 is a mono-carboxy-undecanoate-terminated, monobutyl-terminated polydimethylsiloxane with a molecular weight of 1,500 g/mol that is commercially available from Gelest, Inc. of Morrisville, Pennsylvania, USA. Other carboxyalkyl-terminated polydimethylsiloxanes include bis-carboxypropyl- and bis-carboxydecyl-terminated polydimethylsiloxanes with molecular weights ranging from 1,000 to 28,000, also from Gelest, with tradenames DMS-B12, DMS-B25, and DMS-B31.

Alternatively, (I) the carboxy-functional polyorganosiloxane may be branched. The (I1) branched carboxy-functional polyorganosiloxane may have general formula (I1-1): R^{Car}SiR¹²₃, where R^{Car} has formula

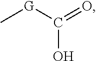

where G is a divalent hydrocarbon group free of aliphatic unsaturation that has 2 to 8 carbon atoms as described above for starting material (E); and each R¹² is selected from R¹³ and —OSi(R¹⁴)₃; where each R¹³ is a monovalent hydrocarbon group; where each R¹⁴ is selected from R¹³, —OSi(R¹⁵)₃, and —[OSiR¹³₂]ᵢᵢOSiR¹³₃; where each R¹⁵ is selected from R¹³, —OSi(R¹⁶)₃, and —[OSiR¹³₂]ᵢᵢOSiR¹³₃; where each R¹⁶ is selected from R¹³ and —[OSiR¹³₂]ᵢᵢOSiR¹³₃; and where subscript ii has a value such that 0≤ii≤100. At least two of R¹² may be —OSi(R¹⁴)₃. Alternatively, all three of R¹² may be —OSi(R¹⁴)₃.

Alternatively, in formula (I1-1) when each R¹² is —OSi(R¹⁴)₃, each R¹⁴ may be —OSi(R¹⁵)₃ moieties such that the branched polyorganosiloxane oligomer has the following structure (I1-2):

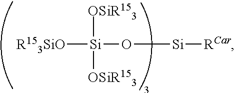

where R^{Car} and R¹ are as described above. Alternatively, each R¹⁵ may be an R¹³, as described above, and each R¹³ may be methyl.

Alternatively, in formula (I1-1), when each R¹² is —OSi(R¹⁴)₃, one R¹⁴ may be R¹³ in each —OSi(R¹⁴)₃ such that each R¹² is —OSiR¹³ (R¹⁴)₂. Alternatively, two R¹⁴ in —OSiR¹³(R¹⁴)₂ may each be —OSi(R¹⁵)₃ moieties such that the branched carboxy-functional polyorganosiloxane oligomer has the following structure (I1-3):

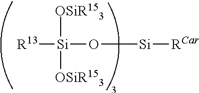

where R^{Car}, R¹³, and R¹⁵ are as described above. Alternatively, each R¹⁵ may be an R¹³, and each R¹³ may be methyl.

Alternatively, in formula (I1-1), one R may be R¹³, and two of R¹² may be —OSi(R¹⁴)₃. When two of R¹² are —OSi(R¹⁴)₃, and one R¹⁴ is R¹³ in each —OSi(R¹⁴)₃ then two of R¹² are —OSiR¹³(R¹⁴)₂. Alternatively, each R¹⁴ in —OSiR¹³(R¹⁴)₂ may be —OSi(R¹⁵)₃ such that the branched polyorganosiloxane oligomer has the following structure (I1-4):

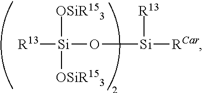

where R^{Car}, R¹³, and R¹⁵ are as described above. Alternatively, each R¹⁵ may be an R¹³, and each R¹³ may be methyl. Alternatively, the carboxy-functional branched polyorganosiloxane may have 3 to 16 silicon atoms per molecule, alternatively 4 to 16 silicon atoms per molecule, alternatively 4 to 10 silicon atoms per molecule, alternatively 7 to 16 silicon atoms per molecule, alternatively 7 to 10 silicon atoms per molecule, and alternatively 10 to 16 silicon atoms per molecule. Examples of carboxy-functional branched polyorganosiloxane oligomers include 3-(1,1,1,5,5,5-hexamethyl-3-((trimethylsilyl)oxy)trisiloxan-3-yl)propanoic acid, which has formula (I1-5):

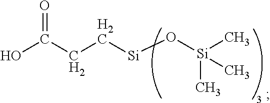

3-(1,1,1,3,5,7,9,9,9-nonamethyl-37-bis((trimethylsilyl)oxy)pentasiloxan-5-yl)propanoic acid, which has formula (I1-6):

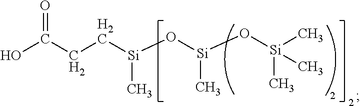

3-(5-((1,1,1,3,5,5,5-heptamethyltrisiloxan-3-yl)oxy)-1,1,1,3,7,9,9,9-octamethyl-3,7-bis((trimethylsilyl)oxy)pentasiloxan-5-yl)propanoic acid, which has formula (I1-7):

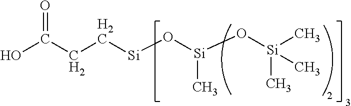

and
7-(5-((1,1,1,3,5,5,5-heptamethyltrisiloxan-3-yl)oxy)-1,1,1,3,7,9,9,9-octamethyl-3,7-bis((trimethylsilyl)oxy)pentasiloxan-5-yl)heptanoic acid, which has formula (I1-8):

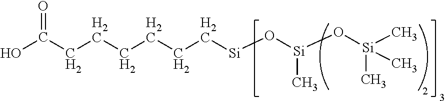

### (Si10Hep-acid).

Alternatively, (I) the carboxy-functional polyorganosiloxane may comprise (I2), a linear polydiorganosiloxane having, per molecule, at least one carboxy-functional group; alternatively at least two carboxy-functional groups. For example, said polydiorganosiloxane may comprise unit formula (I2-1): (R⁴₃SiO_{1/2})ₐ(R^{Car}R⁴₂SiO_{2/2})_{b}(R⁴₂SiO_{2/2})_{c}(R^{Car}R⁴SiO_{2/2})_{d}, where R^{Car} is as described above for formula (I1-1); each R⁴ is as described above for starting material (B1-1); and subscripts a, b, c, and d are as described above for unit formula (B2-1).

Alternatively, the polydiorganosiloxane of unit formula (I2-3) may have formula (I2-2):

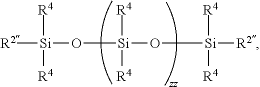

where each R^{2″} is independently selected from the group consisting of R⁴ and R^{Car}, with the proviso that at least one R^{2″} per molecule is R^{Car}, each R^{Car} is as described above for formula (I1-1), each R⁴ is as described above for formula (B1-1), and subscript zz is as described above for formula (B2-2).

Alternatively, this polydiorganosiloxane may comprise two different endgroups, i.e., when subscript a=1 and subscript b=1. Alternatively, this polydiorganosiloxane may be monofunctional, having one carboxy-functional group per molecule, e.g., when subscript a=1, b=1, and d=0 in unit formula (I2-1). This polydiorganosiloxane may have formula (I2-3):

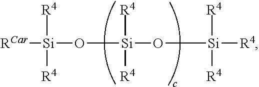

where R^{Car} is as described above for formula (I1-1), each R⁴ is as described above for formula (B1-1), and subscript c is as described above for formula (B2-3).

Alternatively, the linear carboxy-functional polydiorganosiloxane of unit formula (I2-1) may be selected from the group consisting of: unit formula (I2-4): (R⁴₂R^{Car}SiO_{1/2})₂(R⁴₂SiO_{2/2})ₘ(R⁴R^{Car}SiO_{2/2})ₙ, unit formula (I2-5): (R⁴₃SiO_{1/2})₂(R⁴₂SiO_{2/2})ₒ(R⁴R^{Car}SiO_{2/2})ₚ, or a combination of both (I2-4) and (I2-5). In formulae (I2-4) and (I2-5), each R^{Car} is as described above for formula (I1-1), each R⁴ is as described above for formula (B1-1), and subscripts m, n, o, and p are as described above for formulas (B2-4) and (B2-5).

Starting material (I2) may comprise a carboxy-functional polydiorganosiloxane such as i) bis-dimethyl(propanoic acid)siloxy-terminated polydimethylsiloxane, ii) bis-dimethyl(propanoic acid)siloxy-terminated poly(dimethylsiloxane/methyl(propanoic acid)siloxane), iii) bis-dimethyl(propanoic acid)siloxy-terminated polymethyl(propanoic acid)siloxane, iv) bis-trimethylsiloxy-terminated poly(dimethylsiloxane/methyl(propanoic acid)siloxane), v) bis-trimethylsiloxy-terminated polymethyl(propanoic acid)siloxane, vi) bis-dimethyl(propanoic acid)siloxy-terminated poly(dimethylsiloxane/methylphenylsiloxane/methyl(propanoic acid)siloxane), vii) bis-dimethyl(propanoic acid)siloxy-terminated poly(dimethylsiloxane/methylphenylsiloxane), viii) bis-dimethyl(propanoic acid)siloxy-terminated poly(dimethylsiloxane/diphenylsiloxane), ix) bis-phenyl,methyl,(propanoic acid)-siloxy-terminated polydimethylsiloxane, x) bis-dimethyl(heptanoic acid)siloxy-terminated polydimethylsiloxane, xi) bis-dimethyl(heptanoic acid)siloxy-terminated poly(dimethylsiloxane/methyl(heptanoic acid)siloxane), xii) bis-dimethyl(heptanoic acid)siloxy-terminated polymethyl(heptanoic acid)siloxane, xiii) bis-trimethylsiloxy-terminated poly(dimethylsiloxane/methyl(heptanoic acid)siloxane), xiv) bis-trimethylsiloxy-terminated polymethyl(heptanoic acid)siloxane, xv) bis-dimethyl(heptanoic acid)-siloxy terminated poly(dimethylsiloxane/methylphenylsiloxane/methyl(heptanoic acid)siloxane), xvi) bis-dimethyl(propanoic acid)siloxy-terminated poly(dimethylsiloxane/methyl(heptanoic acid)siloxane), xvii) bis-dimethyl(heptanoic acid)-siloxy-terminated poly(dimethylsiloxane/methylphenylsiloxane), xviii) bis-dimethyl(heptanoic acid)-siloxy-terminated poly(dimethylsiloxane/diphenylsiloxane), xix) alpha-dimethyl(n-butyl)siloxy-omega-dimethyl(propanoic acid)siloxy-terminated poly(dimethylsiloxane), and xx) a combination of two or more of i) to xix).

Starting material (I) may be any one of the carboxy-functional organosilicon compounds described above. Alternatively, starting material (I) may comprise a mixture of two or more of the carboxy-functional organosilicon compounds.

### (J) Vinyl Acetate-Functional Compound

In the process for preparing the vinylester-functional polyorganosiloxane, starting material (J) is a vinyl acetate-functional compound. The vinyl-acetate functional compound may have formula vinyl acetate-functional compound of formula 2

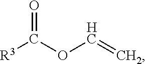

where R³ is an alkyl group of 1 to 6 carbon atoms. Suitable alkyl groups for R³ include methyl, ethyl, propyl (including isopropyl and n-propyl), butyl (including n-butyl, isobutyl, sec-butyl, and t-butyl), pentyl and hexyl, and branched isomers of 5 or 6 carbon atoms. Alternatively starting material (J) may be vinyl acetate, vinyl propionate, vinyl 2-ethylhexanoate, vinyl laurate, and a combination of two or more thereof. Alternatively R³ may be methyl. These vinyl acetate-functional compounds are known in the art and are commercially available from various sources, such as Sigma Aldrich, Inc. of St. Louis, Missouri, USA. Alternatively, starting material (J) may comprise vinyl acetate.

The amount of (J) the vinyl acetate-functional compound may be 0.1 molar equivalents to 20 molar equivalents based on the carboxy-functional group content of (I) the carboxy-functional polyorganosiloxane.

### (K) Transvinylation Reaction Catalyst

The transvinylation reaction catalyst used herein may comprise a metal-ligand complex. The metal may be cobalt (Co), iron (Fe), iridium (Ir), nickel (Ni), osmium (Os), palladium (Pd), platinum (Pt), rhodium (Rh), or ruthenium (Ru). Suitable metal-ligand complexes are described, for example, in Biomacromolecules 2019, 20, 4-26. Alternatively, the transvinylation reaction catalyst may be a palladium-ligand complex. The ligand may be phenanthroline. For example, the transvinylation reaction catalyst may be prepared by a process comprising combining a palladium catalyst precursor and a phenanthroline ligand under conditions to form the complex, which complex may then be introduced into a transvinylation reaction medium comprising one or both of starting materials (I) and/or (J), described above. The palladium catalyst precursor may be any Pd (II) compound. Alternatively, the palladium/phenanthroline complex may be formed in situ by introducing the palladium catalyst precursor into the reaction medium, and introducing the phenanthroline ligand into the reaction medium (e.g., before, during, and/or after introduction of the palladium catalyst precursor), for the in situ formation of the palladium/phenanthroline ligand complex. The palladium/phenanthroline ligand complex can be activated by heating to form the (K) transvinylation reaction catalyst. Palladium catalyst precursors are exemplified by palladium acetate.

For example, a palladium catalyst precursor, such as palladium acetate, optionally starting material (L), the (third) solvent, and the phenanthroline ligand may be combined, e.g., by any convenient means such as mixing. The resulting palladium/phenanthroline ligand complex may be introduced into the reactor, optionally with excess phenanthroline ligand. Alternatively, the palladium catalyst precursor, (L) the solvent, and the phenanthroline ligand may be combined in the reactor with starting material (I) and/or (J), and (K) the transvinylation reaction may form in situ. The relative amounts of phenanthroline ligand and palladium catalyst precursor are sufficient to provide a molar ratio of phenanthroline ligand/Pd of 10/1 to 1/1, alternatively 5/1 to 1/1, alternatively 3/1 to 1/1, alternatively 2.5/1 to 1.5/1.

The amount of (K) the transvinylation reaction catalyst is sufficient to catalyze the transvinylation reaction under the conditions described above. The amount may be 0.0001 mole % to 10 mole % of (K) the transvinylation reaction catalyst based on carboxy-functional groups of (1) the carboxy-functional polyorganosiloxane.

### (L) (Third) Solvent Suitable for Use in Transvinylation Reaction

Starting material (L) is a solvent that may be used in the transvinylation reaction in the process described herein. The solvent may be used to deliver a starting material and/or facilitate the transvinylation reaction. The solvent used for transvinylation reaction may be non protic. The solvent used for transvinylation reaction may be an aliphatic hydrocarbon such as hexane. The amount of (L) the solvent suitable for use in transvinylation reaction is not specifically restricted and may be, for example, 0 to 95 weight % based on combined weights of (I) the carboxy-functional polyorganosiloxane, (J) the vinyl acetate-functional compound, and (K) the transvinylation reaction catalyst.

### (X) Inhibitor

Starting material (X) is an inhibitor that may optionally be added during the transvinylation reaction to minimize or prevent polymerization of the vinylester-functional groups. The inhibitor may comprise a phenolic compound, a quinone or hydroquinone compound, an N-oxyl compound, a phenothiazine compound, a hindered amine compound, or a combination thereof.

Examples of phenolic compounds include phenol, alkylphenols, aminophenols (e.g. p-aminophenol), nitrosophenols, and alkoxyphenols. Specific examples of such phenol compounds include o-, m- and p-cresol(methylphenol), 2-tert-butyl-4-methylphenol, 6-tert-butyl-2,4-dimethylphenol, 2,6-di-tert-butyl-4-methylphenol, 2-tert-butylphenol, 4-tert-butylphenol, 2,4-di-tert-butylphenol, 2-methyl-4-tert-butylphenol, 4-tert-butyl-2,6-dimethylphenol or 2,2′-methylenebis(6-tert-butyl-4-methylphenol), 4,4′-oxybiphenyl, 3,4-methylenedioxydiphenol (sesamol), 3,4-dimethylphenol, pyrocatechol (1,2-dihydroxybenzene), 2-(1′-methylcyclohex-1′-yl)-4,6-dimethylphenol, 2- or 4-(1′-phenyleth-1′-yl)phenol, 2-tert-butyl-6-methylphenol, 2,4,6-tris-tert-butylphenol, 2,6-di-tert-butylphenol, nonylphenol, octylphenol, 2,6-dimethylphenol, bisphenol A, bisphenol B, bisphenol C, bisphenol F, bisphenol S, 3,3′,5,5′-tetrabromobisphenol A, 2,6-di-tert-butyl-p-cresol, methyl 3,5-di-tert-butyl-4-hydroxybenzoate, 4-tert-butylpyrocatechol, 2-hydroxybenzyl alcohol, 2-methoxy-4-methylphenol, 2,3,6-trimethylphenol, 2,4,5-trimethylphenol, 2,4,6-trimethylphenol, 2-isopropylphenol, 4-isopropylphenol, 6-isopropyl-m-cresol, n-octadecyl β-(3,5-di-tert-butyl-4-hydroxyphenyl)propionate, 1,1,3-tris(2-methyl-4-hydroxy-5-tert-butylphenyl)butane, 1,3,5-trimethyl-2,4,6-tris-(3,5-di-tert-butyl-4-hydroxybenzyl)benzene, 1,3,5,-tris(3,5-di-tert-butyl-4-hydroxybenzyl)isocyanurate, 1,3,5-tris(3,5-di-tert-butyl-4-hydroxyphenyl)propionyloxyethyl isocyanurate, 1,3,5-tris(2,6-dimethyl-3-hydroxy-4-tert-butylbenzyl)isocyanurate or pentaerythrityl tetrakis[p-(3,5-di-tert-butyl-4-hydroxyphenyl)propionate], 2,6-di-tert-butyl-4-dimethylaminomethylphenol, 6-sec-butyl-2,4-dinitrophenol, octadecyl 3-(3′,5′-di-tert-butyl-4′-hydroxyphenyl)propionate, hexadecyl 3-(3′,5′-di-tert-butyl-4′-hydroxyphenyl)propionate, octyl 3-(3′,5′-di-tert-butyl-4′-hydroxyphenyl)propionate, 3-thia-1,5-pentanediol bis[(3′,5′-di-tert-butyl-4′-hydroxyphenyl)propionate], 4,8-dioxa-1,11-undecanediol bis[(3′,5′-di-tert-butyl-4′-hydroxyphenyl)propionate], 4,8-dioxa-1,11-undecanediol bis[(3′-tert-butyl-4′-hydroxy-5′-methylphenyl)propionate], 1,9-nonanediol bis[(3′,5′-di-tert-butyl-4′-hydroxyphenyl)propionate], 1,7-heptanediaminebis[3-(3′,5′-di-tert-butyl-4′-hydroxyphenyl)propionamide], 1,1-methanediaminebis[3-(3′,5′-di-tert-butyl-4′-hydroxyphenyl)propionamide], 3-(3′,5′-di-tert-butyl-4′-hydroxyphenyl)propionic acid hydrazide, 3-(3′,5′-dimethyl-4′-hydroxyphenyl)propionic acid hydrazide, bis(3-tert-butyl-5-ethyl-2-hydroxyphen-1-yl)methane, bis(3,5-di-tert-butyl-4-hydroxyphen-1-yl)methane, bis[3-(1′-methylcyclohex-1′-yl)-5-methyl-2-hydroxyphen-1-yl]methane, bis(3-tert-butyl-2-hydroxy-5-methylphen-1-yl)methane, 1,1-bis(5-tert-butyl-4-hydroxy-2-methylphen-1-yl)ethane, bis(5-tert-butyl-4-hydroxy-2-methylphen-1-yl) sulfide, bis(3-tert-butyl-2-hydroxy-5-methylphen-1-yl) sulfide, 1,1-bis(3,4-dimethyl-2-hydroxyphen-1-yl)-2-methylpropane, 1,1-bis(5-tert-butyl-3-methyl-2-hydroxyphen-1-yl)butane, 1,3,5-tris-[1′-(3Δ,5″-di-tert-butyl-4″-hydroxyphen-1″-yl)meth-1′-yl]-2,4,6-trimethylbenzene, 1,1,4-tris(5′-tert-butyl-4′-hydroxy-2′-methylphen-1′-yl)butane and tert-butylcatechol, p-nitrosophenol, p-nitroso-o-cresol, methoxyphenol (guajacol, pyrocatechol monomethyl ether), 2-ethoxyphenol, 2-isopropoxyphenol, 4-methoxyphenol (hydroquinone monomethyl ether, MEHQ), mono- or di-tert-butyl-4-methoxyphenol, 3,5-di-tert-butyl-4-hydroxyanisole, 3-hydroxy-4-methoxybenzyl alcohol, 2,5-dimethoxy-4-hydroxybenzyl alcohol (syringa alcohol), 4-hydroxy-3-methoxybenzaldehyde (vanillin), 4-hydroxy-3-ethoxybenzaldehyde (ethylvanillin), 3-hydroxy-4-methoxybenzaldehyde (isovanillin), 1-(4-hydroxy-3-methoxyphenyl)ethanone (acetovanillone), eugenol, dihydroeugenol, isoeugenol, tocopherols, such as α-, β-, γ-, δ- and ε-tocopherol, tocol, α-tocopherolhydroquinone, 2,3-dihydro-2,2-dimethyl-7-hydroxybenzofuran (2,2-dimethyl-7-hydroxycoumaran), and combinations thereof.

Suitable quinones and hydroquinones include hydroquinone, hydroquinone monomethyl ether(4-methoxyphenol), methylhydroquinone, 2,5-di-tert-butylhydroquinone, 2-methyl-p-hydroquinone, 2,3-dimethylhydroquinone, trimethylhydroquinone, 4-methylpyrocatechol, tert-butylhydroquinone, 3-methylpyrocatechol, benzoquinone, 2-methyl-p-hydroquinone, 2,3-dimethylhydroquinone, tert-butylhydroquinone, 4-ethoxyphenol, 4-butoxyphenol, hydroquinone monobenzyl ether, p-phenoxyphenol, 2-methylhydroquinone, tetramethyl-p-benzoquinone, phenyl-p-benzoquinone, 2,5-dimethyl-3-benzyl-p-benzoquinone, 2-isopropyl-5-methyl-p-benzoquinone (thymoquinone), 2,6-diisopropyl-p-benzoquinone, 2,5-dimethyl-3-hydroxy-p-benzoquinone, 2,5-dihydroxy-p-benzoquinone, embelin, tetrahydroxy-p-benzoquinone, 2,5-dimethoxy-1,4-benzoquinone, 2-amino-5-methyl-p-benzoquinone, 2,5-bisphenylamino-1,4-benzoquinone, 5,8-dihydroxy-1,4-naphthoquinone, 2-anilino-1,4-naphthoquinone, anthraquinone, N,N-dimethylindoaniline, N,N-diphenyl-p-benzoquinonediimine, 1,4-benzoquinone dioxime, 3,3′-di-tert-butyl-5,5′-dimethyldiphenoquinone, p-rosolic acid (aurin), 2,6-di-tert-butyl-4-benzylidenebenzoquinone, 2,5-di-tert-amylhydroquinone, and combinations thereof.

Suitable N-oxyl compounds (i.e., nitroxyl or N-oxyl radicals) include compounds which have at least one N—O● group, such as 4-hydroxy-2,2,6,6-tetramethylpiperidin-N-oxyl, 4-oxo-2,2,6,6-tetramethylpiperidin-N-oxyl, 4-methoxy-2,2,6,6-tetramethylpiperidin-N-oxyl, 4-acetoxy-2,2,6,6-tetramethylpiperidin-N-oxyl, 2,2,6,6-tetramethylpiperidin-N-oxyl (TEMPO), 4,4′,4″-tris(2,2,6,6-tetramethylpiperidin-N-oxyl)phosphite, 3-oxo-2,2,5,5-tetramethylpyrrolidin-N-oxyl, 1-oxyl-2,2,6,6-tetramethyl-4-methoxypiperidine, 1-oxyl-2,2,6,6-tetramethyl-4-trimethylsilyloxypiperidine, 1-oxyl-2,2,6,6-tetramethylpiperidin-4-yl 2-ethylhexanoate, bis(2,2,6,6-tetramethylpiperidin-1-yl)oxyl sebacate, 1-oxyl-2,2,6,6-tetramethylpiperidin-4-yl stearate, 1-oxyl-2,2,6,6-tetramethylpiperidin-4-yl-benzoate, 1-oxyl-2,2,6,6-tetramethylpiperidin-4-yl (4-tert-butyl)benzoate, bis(1-oxyl-2,2,6,6-tetramethylpiperidin4-yl) succinate, bis(1-oxyl-2,2,6,6-tetramethylpiperidin-4-yl) adipate, bis(1-oxyl-2,2,6,6-tetramethylpiperidin-4-yl)1,10-decanedioate, bis(1-oxyl-2,2,6,6-tetramethylpiperidin4-yl)n-butylmalonate, bis(1-oxyl-2,2,6,6-tetramethylpiperidin-4-yl) phthalate, bis(1-oxyl-2,2,6,6-tetramethylpiperidin-4-yl)isophthalate, bis(1-oxyl-2,2,6,6-tetramethylpiperidin4-yl) terephthalate, bis(1-oxyl-2,2,6,6-tetramethylpiperidin-4-yl) hexahydroterephthalate, N,N′-bis(1-oxyl-2,2,6,6-tetramethylpiperidin-4-yl)adipamide, N-(1-oxyl-2,2,6,6-tetramethylpiperidin-4-yl)caprolactam, N-(1-oxyl-2,2,6,6-tetramethylpiperidin-4-yl)dodecylsuccinimide, 2,4,6-tris[N-butyl-N-(1-oxyl-2,2,6,6-tetramethylpiperidin-4-yl]triazine, N,N′-bis(1-oxyl-2,2,6,6-tetramethylpiperidin-4-yl)-N,N′-bisformyl-1,6-diaminohexane, 4,4′-ethylenebis(1-oxyl-2,2,6,6-tetramethylpiperazin-3-one), and combinations thereof.

Other compounds suitable for use in or as the inhibitor include phenothiazine (PTZ) and compounds with similar structures, such as phenoxazine, promazine, N,N′-dimethylphenazine, carbazole, N-ethylcarbazole, N-benzylphenothiazine, N-(1-phenylethyl)phenothiazine, N-Alkylated phenothiazine derivatives such as N-benzylphenothiazine and N-(1-phenylethyl)phenothiazine, and the like. Alternatively, the inhibitor may be selected from the group consisting of 4-methoxyphenol (MEHQ), (2,2,6,6-tetramethylpiperidin-1-yl)oxyl (TEMPO), 4-hydroxy (2,2,6,6-tetramethylpiperidin-1-yl)oxyl (4HT), bis(2,2,6,6-tetramethylpiperidin-1-yl)oxyl sebacate (Bis-TEMPO), polymer-bound TEMPO, or a combination thereof.

The inhibitor may be used to prevent polymerization of the vinylester-functional group before use of a starting material (e.g., starting material (J)) and/or during the transvinylation reaction, and/or after the transvinylation reaction. The amount of (X) the inhibitor depends on various factors including the type and amount of (J) the vinylacetate-functional compound, however, (X) the inhibitor may present in an amount of 10 ppm to 10,000 ppm, alternatively 50 ppm to 1,000 ppm, based on weight of (J) the vinyl-acetate functional compound and/or based on weight of (M1) the vinylester-functional siloxane macromonomer (e.g., if added after the transvinylation reaction).

The transvinylation reaction process described above can be used to produce the vinylester-functional siloxane macromonomer useful as starting material M1) (described above) in the process for making the copolymer described herein. Starting material M1) is used in the mixture of monomers with M2) the alkenyl ester of the aliphatic fatty acid and optionally M3) the additional monomer, introduced above and described in detail below.

### Starting Material M2) Vinyl Ester of Aliphatic Fatty Acid

Starting material M2) used in the process for preparing the silicone-vinylester copolymer is a vinylester of an aliphatic fatty acid. Starting material M2) may have formula:

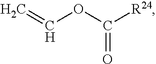

where R²⁴ is hydrogen or an alkyl group of 1 to 14 carbon atoms. The alkyl group for R²⁴ may be linear or branched. For example, the alkyl group may be methyl, ethyl, propyl (including n-propyl and/or isopropyl), butyl (including n-butyl, isobutyl, sec-butyl, and/or tert-butyl), pentyl, hexyl, heptyl, octyl, nonyl, decyl, undecyl, dodecyl, tridecyl, and tetradecyl (and saturated, branched isomers having 5 to 14 carbon atoms). Alternatively, R²⁴ may be methyl. Alternatively, starting material M2) may comprise vinyl acetate, which is commercially available from Sigma-Aldrich, Inc. of St. Louis, Missouri, USA.

The amount of M2) the vinylester of the aliphatic fatty acid in the mixture of monomers depends on various factors including the selection and amount of each other monomer in the mixture and the desired end use of the copolymer. However, the amount of M2) the vinylester of the aliphatic fatty acid may be 1% to 99%, alternatively 40% to 70%, based on combined weights of all monomers in the mixture of monomers (e.g., based on combined weights of M1), M2), and M3), described herein).

### Starting Material M3) Optional Additional Monomer

The mixture of monomers described above may optionally further comprise an additional ethylenically unsaturated monomer that differs from starting materials M1) and M2), described above, but that can be copolymerized with starting materials M1) and M2). The optional additional monomer may be a (meth)acrylic monomer of formula:

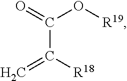

where R¹⁸ is hydrogen or a methyl group, and R¹⁹ is hydrogen or an alkyl group of 1 to 22 carbon atoms, where the alkyl group may be linear or branched. The alkyl group for R¹⁹ is exemplified by methyl, ethyl, propyl (including n-propyl and isopropyl), butyl (including n-butyl, isobutyl, tert-butyl, and sec-butyl); pentyl, hexyl, heptyl octyl, nonyl, decyl, undecyl, dodecyl, tridecyl, and tetradecyl (and branched alkyl groups of 5 to 14 carbon atoms). Alternatively R¹⁸ may be hydrogen. Alternatively R¹⁹ may be an alkyl group of 1 to 14 carbon atoms, alternatively 1 to 12 carbon atoms, alternatively 1 to 10 carbon atoms, alternatively 1 to 6 carbon atoms, alternatively 1 to 4 carbon atoms, and alternatively 1 to 2 carbon atoms.

Alternatively, the optional additional monomer may be a cyclic ketene acetal monomer. The cyclic ketene acetal monomer may be as described in U.S. Patent Application Publication 2020/0325261 to Carter, et al. The cyclic ketene acetal monomer may have formula:

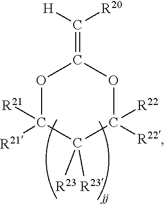

where subscript jj is 0, 1, or 2; R²⁰ is hydrogen or an alkyl group of 1 to 6 carbon atoms, R²¹ and R²² are each independently selected from hydrogen, an alkyl group of 1 to 12 carbon atoms, phenyl, or vinyl, with the proviso that R²1 and R²², together with the carbon atoms to which they are attached form a fused benzene ring or a fused cycloaliphatic ring with 3 to 7 carbon atoms; R^{21′} and R^{22′} are each independently selected from hydrogen or an alkyl group of 1 to 12 carbon atoms, with the provisos that R²¹ and R^{21′} and/or R²² and R^{22′} can form an exocyclic double bond. Examples of cyclic ketene acetal monomers where jj=2 in the formula above include 4,7-dimethyl-1,2-methylene-1,3-dioxepane; 2-methylene-1,3-dioxepane (MDO); 2-methylene-4,7-dihydro-1,3-dioxepine; and 3-methylene-1,5-dihydrobenzo[e][1,3]dioxepine; and a combination thereof. Examples of cyclic ketene acetal monomers where jj=1 in the formula above include 4-methyl-2-methylene-1,3-dioxane; 4,6-dimethyl-2-methylene-1,3-dioxane; 5,5-dimethyl-2-methylene-1,3-dioxane; 2-methylene-1,3-dioxane; 2-methylene-4-phenyl-1,3-dioxane; 3,9-dimethylene-2,4,8,10-tetraoxaspiro[5.5]undecane; and a combination thereof. Examples of cyclic ketene acetal monomers where jj=0 in the formula above include 2-methylene-1,3-dioxolane; 4-methyl-2-methylene-1,3-dioxolane; 4-hexyl-2-methylene-1,3-dioxolane; 4-decyl-2-methylene-1,3-dioxolane; 4,5-dimethyl-2-methylene-1,3-dioxolane; 2-methylenehexahydrobenzo[d][1,3]dioxole; and a combination thereof.

Alternatively, M3) the optional additional monomer may be selected from the group consisting of acrylic acid, MDO, and a combination thereof. Suitable additional monomers are known in the art and are commercially available, e.g., from Sigma-Aldrich, Inc.

The amount of M3) the additional monomer that may optionally be included in the mixture of monomers depends on various factors including the selection and amount of other monomers in the mixture and the desired end use of the copolymer. However, the amount of M3) the additional monomer may be 0 to 20%, alternatively 0 to 10%, based on combined weights of all monomers in the mixture of monomers (e.g., based on combined weights of M1), M2), and M3), described herein).

### Method for Preparing the Copolymer

The silicone-vinylester copolymer (introduced above) may be prepared by a method comprising: I) combining, under conditions to conduct free radical polymerization, starting materials comprising: the mixture of monomers comprising M1), M2), and optionally M3), described above, N) a radical initiator, and optionally O) a (fourth) solvent (i.e., a solvent suitable for use in the copolymerization reaction of the mixture of monomers); thereby forming a reaction mixture. The method may further comprise II) quenching the reaction mixture after step I). The method may optionally further comprise III) recovering the copolymer form the reaction mixture, and/or IV) dissolving the copolymer in a simple alcohol such as ethanol, and/or V) performing a solvent exchange to dissolve the copolymer in P) a carrier suitable for use in personal care compositions.

In step I), the mixture of monomers comprising M1), M2), and when present, M3), as described above may be copolymerized by mixing and heating to form a reaction mixture. The copolymerization may be performed by a free radical polymerization, and N) the radical initiator may be combined with the mixture of monomers in step I).

### N) Radical Initiator

Starting material N), the radical initiator may be, for example, an azo-based compound, an organic peroxide, or a combination thereof. The radical initiator may comprise, for example, an azo-based compound such as 2,2′-azobis(isobutyronitrile); 2,2′-azobis(2-methyl)butyronitrile; 2,2′-azobis(2,4-dimethylvaleronitrile); dimethyl-2,2′-azobis(2-methyl propionate); and a combination of two or more thereof. Alternatively, the radical initiator may comprise, for example, an organic peroxide such as benzoyl peroxide, lauroyl peroxide, tert-butyl peroxybenzoate; tert-butyl peroxy-2-ethylhexanoate, tert-amyl peroxypivalate, cyclohexanone peroxide, isopropyl cumyl hydroperoxide, di-tert-butyl peroxide, diisopropyl percarbonate, tert-butyl perbenzoate, tert-butyl peroctanoate, bis(3,5,5-trimethyl)hexanoyl peroxide, tert-butylperoxypivalate, and combinations of two or more thereof. The radical initiators are known in the art and are commercially available. For example, organic peroxides such as peroxydicarbonates, diacylperoxides, dialkyl peroxides, and peroxyeters are available under the tradename LUPEROX™ from Arkema Inc. of King of Prussia, Pennsylvania, USA. Tert-amylperoxypivalate is available from AkzoNobel under the tradename TRIGONOX™. The amount of radical initiator may be 0.1 weight parts to 5 weight parts, per 100 parts by weight of the mixture of monomers used in step I). Other examples of initiators suitable for the free-radical polymerization of these vinyl ester monomers can be found in the product brochure titled “Initiators for acrylics manufacturing” by AkzoNobel published in 2018, and the product brochure titled “azo polymerization initiators comprehensive catalog” by Wako Pure Chemical Industries, Ltd.

The copolymerization in step I) may be a solution polymerization, and O) a solvent suitable for use in the radical polymerization reaction may be added in step I). One or more of the starting materials (e.g., N) the radical initiator and/or a mixture of M1) the macromonomer and M2) alkenyl ester of aliphatic fatty acid and optionally M3)) may be delivered in the solvent. For example, the radical initiator may be delivered in mineral spirits. The solvent for solution polymerization may be in addition to, or instead of, the solvent for delivery of a starting material. The solvent in step I) may comprise a simple alcohol of formula R²OH, where R² is a monovalent hydrocarbon group of 1 to 4 carbon atoms, alternatively an alkyl group of 1 to 4 carbon atoms. The simple alcohol is exemplified by ethanol, n-propanol, isopropanol, n-butanol, t-butanol, or a combination thereof. The simple alcohol may comprise ethanol. Alternatively, the simple alcohol may be selected from ethanol, isopropanol, or a combination thereof. Alternatively, the solvent in step I) may comprise a simple aliphatic ester of formula R²O(CO)R⁵, where R² is as defined above, and R^{S} is hydrogen or an alkyl group of 1 to 14 carbon atoms (as described above for R³). Alternatively, the simple aliphatic ester may be ethyl acetate. Alternatively, the solvent in step I) may be a combination of one or more simple alcohols of formula R²OH and one or more simple aliphatic esters of formula R²O(CO)R⁵.

Step I) may be performed for example in a batch, semi-batch, or continuous mode over 3 hours to 20 hours at a temperature from >30° C., alternatively 50° C. to 150° C. In the semi-batch mode, a fraction of the reagents may be dosed into the reactor (e.g., the radical initiator, the mixture of monomers, and/or the solvent in which the radical initiator and the mixture of monomers is delivered), and the remainder of the reagents are metered into the reactor over a targeted feed time, typically 1 hour to 20 hours. In the continuous mode, the reagents are continuously metered into the reactor, and the reaction mixture comprising the copolymer is continuously withdrawn from the reactor. The starting materials used in step I) may be free of acids.

In step II), quenching may be performed by cooling the reaction mixture to 25±5° C.

The method may optionally further comprise step III) recovering the copolymer from the reaction mixture. Recovery of the copolymer can be carried out by any convenient means. For example, if any unreacted monomer or monomers are present, and/or solvents are used, these may be removed, e.g., by heating, optionally with reduced pressure. For example, stripping and/or distillation may be used to purify the copolymer. Alternatively, the unreacted monomers may be reduced by chemical chase, in which an additional radical initiator is added to consume the unreacted monomers and bring the level down to sufficiently low level (e.g., <500 ppm) that the unreacted monomers do not need to be removed.

The method may optionally further comprise IV) dissolving the copolymer in a simple alcohol such as ethanol, for example, when solvent is not used in step I).

Alternatively, the method may optionally further comprise step V) solvent exchange after II) or after step III) or after step IV), when present. If P) a different carrier for the copolymer is desired, O) the solvent described above may be removed and replaced with the different carrier, such as a personal care friendly carrier. Alternatively, P) the carrier may be selected from the group consisting of alcohols including monohydric alcohols such as ethanol, isopropyl alcohol, n-propanol, tert-butanol, and sec-butanol, polyhydric alcohols including dihydric alcohols such as 1,3-propanediol, 1,3-butylene glycol, 1,2-butylene glycol, propylene glycol, trimethylene glycol, tetramethylene glycol, 2,3-butylene glycol, pentamethylene glycol, 2-butene-1,4-diol, dibutylene glycol, pentyl glycol, hexylene glycol, and octylene glycol, trihydric alcohols such as trimethylolpropane, and 1,2,6-hexanetriol, tetrahydric alcohols and higher such as pentaerythritol and xylitol, sugar alcohols such as sorbitol, ketones including acetone and methyl ethyl ketone, fatty acid esters including isopropyl myristate and isopropyl palmitate, natural oils including sunflower seed oil, caprylic/capric triglycerides, coconut oil, castor oil, argan oil, and jojoba oil, hydrocarbon oils including isododecane, isohexadecane, paraffin, isoparaffin, squalane, and squalene, and an alkane of 9 to 11 carbon atoms, siloxane or silicone oils such as decamethylcyclopentasiloxane (D5) and linear low-viscosity polydimethylsiloxane (PDMS) (e.g., with viscosity of 1.5 cSt to 6 cSt at 25° C. by rotational viscometry, using a Brookfield viscometer), glycerin; glycerol; or a combination of two or more thereof. Such carriers are commercially available. For example, D5 and linear polydimethylsiloxanes with the viscosities above are available from Dow Silicones Corporation, Midland, Michigan, USA.

### Copolymer

The copolymer prepared as described above may comprise unit formula:

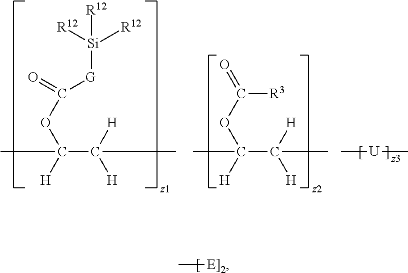

where R¹², G, and R³ are as described above; subscripts z1, z2, and z3 represent weight factions, z1 is 0.01 to 0.99, z2 is 0.01 to 0.99, and z3 is 0 to 0.2, with the proviso that a quantity (z1+z2+z3)=1; U represents a unit derived from starting material M3); and E is an endblocking moiety. Alternatively, z1 may be 0.3 to 0.6. Alternatively, z3 may be 0.4 to 0.7. Alternatively, z3 may be 0 to 0.1.

The unit derived from starting material M3) may have formula:

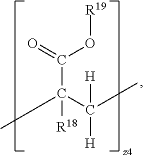

when the (meth)acrylic monomer described above is used. In this formula, R¹⁸ and R¹⁹ are as described above for the (meth)acrylic monomer. And, subscript z4 may be equal to z3 when no other additional monomers are used as starting material M3).

Alternatively, the unit derived from starting material M3) may have formula:

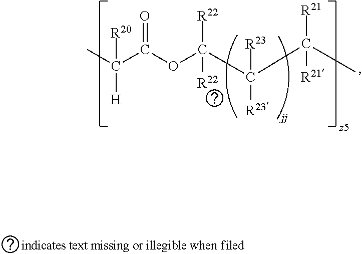

when the cyclic ketene acetal monomer is used. In this formula, R²⁰, R²¹, R^{21′}, R²², R^{22′}, R²³, R^{23′}, and subscript jj are as described above for the cyclic ketene acetal monomer. And, subscript z5 may be equal to z3 when no other additional monomers are used as starting material M3).

The copolymer further comprises an end unit. The end units of the copolymers are fragments of initiators and/or hydrogen atoms. Examples of suitable initiators are as described above and include t-amyl peroxypivalate (commercially available as Trigonox 125-C75 initiator), t-butyl peroxypivalate (commercially available as Trigonox 25-C75), t-amyl peroxy-2-ethylhexanoate; 2,2′-azobis(2-methylbutyronitrile), and dimethyl 2,2′-azobis(2-methyl propionate).

The copolymer described herein may have glass transition temperature (measured by DSC as described in the EXAMPLES, below) that varies depending on various factors including the amount of each of starting materials M1) and M2) and whether an additional monomer M3) is present, however glass transition temperature may be −20° C. to 25° C., alternatively 0° C. to 20° C., alternatively 3° C. to 19° C., and alternatively 0° C. to 5° C.

The molecular weight and polydispersity of the copolymer described herein depends on various factors including the number of silicon atoms per molecule in M1) the macromonomer, the amounts of each of M1) and M2) the vinylester of the aliphatic fatty acid, and whether M3) the additional monomer is present. However, molecular weight (i.e., number average molecular weight and weight average molecular weight) may be measured by GPC as described in the examples below. The copolymer may have Mn of 4 kg/mol to 20 kg/mol, alternatively 5 kg/mol to 18 kg/mol, alternatively 6 kg/mol to 16 kg/mol. The copolymer may have Mw of 15 kg/mol to 100 kg/mol, alternatively 16 kg/mol to 75 kg/mol, alternatively 17 kg/mol to 70 kg/mol, and alternatively 18 kg/mol to 55 kg/mol.

### Method of Use

The copolymer prepared as described herein may be added into a personal care composition. For example, the copolymer described above may act as a film forming agent in a personal care composition. The personal care composition is not specifically restricted, however, the personal care composition may be a leave-on product suitable for application to the skin, such as skin care, sunscreen, and color cosmetic products (e.g., a foundation). The copolymer, or solution of copolymer, prepared as described above may be added to the personal care composition by any convenient means, such as mixing. The personal care composition may comprise the copolymer described above in any amount, e.g., at least 1%, alternatively at least 2%, alternatively at least 5%, alternatively at least 10%, alternatively at least 20%, and alternatively at least 30%, based on weight of all components of the personal care composition (and excluding the carrier for delivery of the copolymer). At the same time, the personal care composition may comprise the copolymer described above in an amount of up to 99%, alternatively up to 90%, alternatively up to 80%, alternatively up to 70%, alternatively up to 50%, alternatively up to 10%, alternatively up to 8%, and alternatively up to 6%, based on weight of all components of the personal care composition (and excluding the carrier for delivery of the copolymer). The exact amount of copolymer used depends on various factors, such as the type of personal care composition to be formulated. Alternatively, the personal care composition may contain the copolymer in an amount of 1 weight % to 99 weight %, alternatively 5% to 95%, alternatively 1% to 10%, alternatively 2% to 8%, and alternatively 4% to 6%, based on weight of all components of the personal care composition (and excluding the carrier for delivery of the copolymer).

The copolymer prepared as described above, may be used in place of the different copolymers in personal care compositions known in the art, such as those disclosed in U.S. Pat. No. 7,488,492 to Furukawa, et al.; U.S. Pat. No. 9,670,301 to Furukawa, et al.; U.S. Pat. No. 10,047,199 to Iimura, et al.; U.S. Pat. No. 10,172,779 to Hori, et al.; and U.S. Patent Application Publication 2020/0222300 to Souda, et al. Alternatively, the copolymer described herein may be formulated into the sunscreens and cosmetics described in U.S. Patent Application Publication 2019/0201317 to Konishi, et al., in place of the organosiloxane graft polyvinyl alcohol polymer described therein. Alternatively, the copolymer described herein may be used in the cosmetic raw material of U.S. Pat. No. 6,280,748 to Morita, et al. instead of the (meth)acrylate functional carbosiloxane dendrimer disclosed therein. Alternatively, the copolymer described herein may be formulated into the suncare and cosmetic compositions of U.S. Pat. No. 8,541,009 to lida, et al. instead of the (meth)acrylate based polymer having a carbosiloxane dendrimer structure in a side chain thereof. Alternatively, the copolymer described herein may be formulated int o the make-up and/or care composition for keratin materials of U.S. Pat. No. 8,828,372 to Arnaud, et al. in place of the vinyl polymer comprising carbosiloxane dendrimer derived units.

### EXAMPLES

These examples are intended to illustrate the invention to one of ordinary skill in the art and are not to be construed to limit the scope of the invention set forth in the claims. Starting materials used in these examples are described in Table 1.

TABLE 1
STARTING MATERIALS

| | | | |
|---|---|---|---|
| Starting Material | | Chemical Description, Chemical | |
| Type | Product Name | formula, or Structure | Source |

Starting Materials for macromonomer synthesis

| | | | |
|---|---|---|---|
| Starting material | Si10Vi | Shown above | Dow |
| Starting material | Si10-Hexenyl | Shown above | Dow |
| Starting Material | monovinyl terminated | ViSiMe₂(OSiMe₂)₃OSiMe₃ | Gelest |
| | polydimethylsiloxane | (Mw = 5500-6500 g/mol) | |
| | (MCR-V21) | | |
| Starting Material | 1,1,1,5,5,5- | ViSi(OSiMe₃)₃ | DSC |
| | hexamethyl-3- | | |
| | ((trimethylsilyl)oxy)-3- | | |
| | vinyltrisiloxane | | |
| | (Si4Vi) | | |
| Hydroformylation | Rh(acac)(CO)₂ | (Acetylacetonato)dicarbonylrhodium(I) | Sigma- |
| Catalyst | | | Aldrich |
| precursor | | | |
| Catalyst | Palladium acetate | | Sigma- |
| | | | Aldrich |
| Catalyst | 1,10-Phenanthroline | | Sigma- |
| | | | Aldrich |
| Ligand 1 | 6,6′-[[3,3′,5,5′- | | Dow |
| | tetrakis(1,1- | | |
| | dimethylethyl)-1,1′- | | |
| | biphenyl]-2,2′- | | |
| | diyl]bis(oxy)bis- | | |
| | dibenzo[d,f] | | |
| | [1,3,2]dioxaphosphepin | | |
| Gas | Syngas | CO/H₂, 50/50 (mol %) | Airgas |
| | | | U.S.A. LLC |
| Inhibitor | MEHQ | 4-Methoxyphenol | Sigma- |
| | | | Aldrich |

Starting Materials for copolymer synthesis

| | | | |
|---|---|---|---|
| EtOAc, Solvent | Ethyl acetate | CH₃COOCH₂CH₃ | Sigma- |
| | | | Aldrich |
| Solvent | Ethanol | CH₃CH₂OH | Dow |
| VAc, Monomer | Vinyl acetate | CH₃COOCH═CH₂ | Dow |
| MDO, Monomer | 2-methylene-1,3- dioxepane (MDO) (CAS #69814-56-8) | 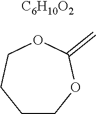 | Wacker |
| Monomer | Acrylic acid | | Dow |
| Initiator Solution | tert-amyl | Trigonox 125-C75 | Akzo |
| | peroxypivalate (CAS | | Nobel |
| | #29240-17-3), 75% | | |
| | active in mineral spirits | | |

Starting Materials for foundation formulations

| | | | |
|---|---|---|---|
| Carrier | Permethyl 99A | isododecane | Presperse |
| Pigment | SAT-B-335198 | Iron oxide and dimethicone | Miyoshi |
| Pigment | SAT-R-338075 | Iron oxide and dimethicone | Miyoshi |
| Pigment | SAT-Y-338073 | Iron oxide and dimethicone | Miyoshi |
| Pigment | SAT-TRI-77891 | Titanium dioxide (and) dimethicone | Miyoshi |
| Emollient | DOWSIL ™ 3196 | Caprylyl Methicone | Dow |
| Formulation aid | DOWSIL ™ 5300 | Lauryl PEG-10 | Dow |
| | | Tris(trimethylsiloxy)silylethyl | |
| | | Dimethicone | |
| Formulation aid | Sodium chloride | Sodium chloride | Sigma- |
| | | | Aldrich |
| Carrier | Glycerin | Glycerin | Sigma- |
| | | | Aldrich |
| Preservative | Euxyl ™ PE 9010 | Phenoxyethanol | Schülke & |
| | | (and) | Mayr |
| | | ethylhexyglycerin | GmbH |

Starting Materials for sunscreen formulations

| | | | |
|---|---|---|---|
| Sunscreen active | Avobenzone | Butyl Methoxydibenzoylmethane | Making |
| | | | Cosmetics |
| Sunscreen active | Octisalate | Ethylhexyl Salicylate | Spectrum |
| Sunscreen active | Octocrylene | Octocrylene | Making |
| | | | Cosmetics |
| Sunscreen active | Homosalate | Homosalate | Making |
| | | | Cosmetics |
| Solvent | DI Water | Water | Local |
| Solvent, IDD | Permethyl 99A | Isododecane | Presperse |
| Solvent | Ritamollient | C12-15 Alkyl Benzoate | Rita |
| Solvent | Xiameter PMX-200, 2 | Dimethicone | Dow |
| | cst | | |
| Rheology | Aculyn 38 | Acrylates/Vinyl Neodecanoate | Dow |
| modifier | | Crosspolymer | |
| Chealant | Versene NA2 Crystal | Disodium EDTA | Dow |
| Salt | Sodium Chloride | Sodium Chloride | Fisher |
| | | | Scientific |
| Emollient | Zemea | 1,3-Propanediol | DuPont |
| | | | Tate & Lyle |
| Emollient | DOWSIL ™ 2502 | Cetyl Dimethicone | Dow |
| | Cosmetic Fluid | | |
| Emulsifier | DOWSIL ™ ES-5226 | Dimethicone (and) PEG/PPG-18/18 | Dow |
| | DM Formulation Aid | Dimethicone | |
| Emulsifier | Ritox 52 | PEG-40 Stearate | Rita |
| Emulsifier | Rita GMS | Glyceryl Stearate | Rita |
| Emulsifier | Procol CS-20-D | Cetearyl Alcohol (and) Cetereth-20 | Protameen |
| pH adjuster | Sodium Hydroxide | Sodium Hydroxide | Fisher |
| | | | Scientific |
| Preservative | Phenoxyethanol | Phenoxyethanol | Spectrum |
| Film former | DOWSIL ™ FA 4012 | Isododecane, Acrylates Polytrimethyl | Dow |
| | IDD | siloxymethacrylate Copolymer | |

### Example 1—Hydroformylation of Si10-Hexenyl

In this Example 1, hydroformylation of a Si10-Hexenyl branched oligomer was performed as follows: In a nitrogen filled glovebox, Rh(acac)(CO)₂ (25.5 mg, 0.0984 mmol), Ligand 1 (122.3 mg, 0.1457 mmol), and toluene (5.0 g) were added into a 30 mL vial with a magnetic stir bar. The mixture was mixed on a magnetic stir plate until a homogeneous solution formed. The solution was transferred to an air-tight syringe with a metal valve and removed from the glove box. In a fume hood, Si10-Hexenyl (100.0 g, 121.6 mmol) was added to the Parr reactor. The reactor was sealed and loaded into the holder. The reactor was pressurized with nitrogen up to 100 psi (690 kPa) via the dip-tube and was carefully released through headspace for three times. After pressure testing, the catalyst solution was added to the reactor. The reactor was pressurized with syngas to 100 psi and then released for three times prior to being pressurized to 80 psi via the dip-tube. Agitation and heating were initiated. The intermediate cylinder containing syngas and the reactor were connected when the reaction reached 110° C. The pressure of the intermediate cylinder was monitored by a data logger. After the reaction was done, the reactor was purged with nitrogen for three times and the material was transferred to a glass container as a colorless liquid, which turned light yellow over time. The product was crude (M2T)3T Heptanaldehyde (Si10HeptAld).

### Example 2—Oxidation of Si10-Heptanaldehyde

In this Example 2, oxidation of (M2T)3T Heptanaldehyde was performed as follows: Crude (M2T)3T Heptanealdehyde (100 g) which contained approximately 5 wt % toluene (as prepared in Example 1) was loaded to a one-neck round bottom flask equipped with a PTFE coated stir bar and a septa cap. The liquid was stirred on a magnetic stir plate at 1150 rpm as air was sparged subsurface through a needle. The reaction mixture was analyzed by ¹H NMR until complete. The reaction was stopped after 24 hours and the (M2T)3T-Heptanoic acid product (Si10Heptanoic acid) was collected as a clear orange liquid.

### Example 3—Transvinylation of Si10-Heptanoic Acid

In this Example 3, a three-neck round bottom flask equipped with a thermometer, a condenser with a bubbler on top and a rubber septum was used for this synthesis. A heating mantle with a J-Kem controller was used for controlling the heating. A magnetic stir bar, (M2T)3T-Heptanoic acid product prepared as described in Example 2, above, (176.0 g, 0.2026 mol) and vinyl acetate (186.0 g, 2.163 mol) were added to the reactor. Nitrogen was bubbled into the mixture subsurface through a needle under vigorous stirring for 5 minutes. Palladium acetate (0.2390 g, 0.001067 mol) and phenanthroline (0.4070 g, 0.002258 mol) were added via the septa port. This mixture was stirred for 10 minutes before the reaction mixture was heated to 60° C. overnight. After the reaction was done, materials were evaporated on a rotary evaporator with a bath temperature of 40° C. (500-10 mbar). 200 mL of hexane was added to the flask. The mixture was filtered through a disposable filter with 7 g silica. The filtrate was concentrated under a rotary evaporator, affording a yellow viscous liquid (165.0 g, 96% purity). MEHQ (60.3 mg) was added to the material. The resulting material was Si10HepVE, which was stored in a brown bottle until further use.

### Example 4—Hydroformylation of Si10Vi

In this Example 4, In a nitrogen filled glovebox, Rh(acac)(CO)₂ (15.8 mg, 0.0610 mmol), Ligand 1 (75.1 mg, 0.0895 mmol) and toluene (7.5 g) were added into a 30 mL vial with a magnetic stir bar. The mixture was mixed on a magnetic stir plate until a homogeneous solution was formed. The solution was transferred to an air-tight syringe with a metal valve and removed from the glove box. In a fume hood, Si10Vi (142.4 g, 185.8 mmol) was added to the Parr reactor. The reactor was sealed and loaded into the holder. The reactor was pressurized with nitrogen up to 100 psi via the dip-tube and was carefully released through headspace for three times. After pressure testing, the catalyst solution was added to the reactors. The reactor was pressurized with syngas to 100 psi and then released for three times prior to being pressurized to 80 psi via the dip-tube. Agitation and heating were initiated. The intermediate cylinder containing syngas and the reactor were connected when the reaction reached 100° C. The pressure of the intermediate cylinder was monitored by a data logger. After the reaction was done, the reactor was purged with nitrogen for three times and the material was transferred to a glass container as a colorless liquid, which turned light yellow over time. This material was labeled crude Si10PrAld.

### Example 5—Oxidation of Si10-propanaldehyde

In this Example 5, crude Si10PrAld prepared as described above in Example 4 (51.92 g) which contained approximately 5 wt % toluene was loaded to an 8-ounce narrow mouth glass bottle equipped with a PTFE coated stir bar and a septa cap. The liquid was stirred on a magnetic stir plate at 900 rpm as air was sparged subsurface through a needle at 100 cc/min. The reaction mixture was analyzed by ¹H NMR until complete. The reaction was stopped after 24 hours and the resulting Si10PrAcid product was collected (50.5 g) as a clear slightly yellow liquid.

### Example 6—Transvinylation of Si10-Propanoic Acid

In this Example 6, a three-neck round bottom flask equipped with a thermometer, a condenser with a bubbler on top and a rubber septum was used for this synthesis. A heating mantle with a J-Kem controller was used for controlling the heating. A magnetic stir bar, Si10PrAcid prepared as described above in Example 5 (110.0 g, 0.1354 mol) and vinyl acetate (129.1 g, 1.501 mol) were added to the reactor. Nitrogen was bubbled into the mixture subsurface through a needle under vigorous stirring for 5 minutes. Palladium acetate (0.3406 g, 0.001520 mol) and phenanthroline (0.4273 g, 0.002371 mol) were added via the septa port. This mixture was stirred for 10 minutes before the reaction mixture was heated to 60° C. overnight. After the reaction was done, materials were transferred to a round bottom flask and evaporated on a rotary evaporator with a bath temperature of 40° C. (500-10 mbar). 200 mL of hexane was added to the flask. The mixture was filtered through a disposable filter with 7 g of silica. The filtrate was concentrated under a rotary evaporator, affording a yellow viscous liquid. The macromonomer prepared by this example 6 was labeled Si10PrVi.

### Comparative Example 7—Synthesis of Solution Polymer by Free-Radical Polymerization

In this comparative example 7, a polymer designated CE P1 was prepared via solution polymerization in a thermal, semi-batch process. The polymer backbone was made of 100% vinylacetate (Vac). A 500-mL round-bottom flask was equipped with a glass rod propeller connected with a half-moon Teflon blade, a condenser, and a thermocouple. The propeller was driven by an overhead mechanical stirrer, and the thermocouple was connected with a J-KEM temperature controller and provided input to a pneumatic potlifter to achieve the desired temperature. The flask was first charged with 45.0 g of EtOAc and heated to 65° C. A N₂ blanket was applied to remove the entrained air, and the agitation rate started at 120 rpm. A separate 8 oz glass jar was charged with 60.0 g of VAc monomer. The cofeed initiator was prepared by diluting 0.80 g of Trigonox 125-C75 in 22.50 g of EtOAc. When the temperature of the reactor reached 50° C., 12 g of the VAc monomer was transferred into the reactor and heat continued to be applied. When the reactor temperature reached 65° C., the rest of the VAc monomer and the cofeed initiator started to be metered in at the rate of 0.40 g/min and 0.19 g/min, respectively. The targeted feed time was 120 min. Moderate heat continued to be applied to maintain the polymerization temperature at 65° C. When the monomer feed was completed, 6.00 g of EtOAc was added into the monomer jar and rinsed into the reactor. The batch was held at 65° C. for 30 min. Then two chemical chases of 1.20 g of Trigonox 125-C75 in 4.50 g of EtOAc were metered in at a rate of 0.19 g/min over 30 minutes each with a 15-minute hold in between. The temperature increased to 70° C. during the chemical chases. The batch was held for another 60 min before the reaction was quenched by cooling to ambient temperature.

The resulting polyvinylacetate polymer was a solid 40 wt % dissolved in EtOAc. Residual VAc was below the detection limit (typically deemed as 200 ppm) by ¹H NMR spectroscopy. The Mₙ, M_{w}, and dispersity were 19.3 kg/mol, 84.3 kg/mol, and 4.37, respectively (relative to polystyrene (PS) standards). THF with 0.1 wt % of formic acid was the mobile phase of the GPC. T_{g} was 16.7° C. by DSC using the 2^{nd} heating at a rate of 20° C./min.

### Example 8—Copolymer Synthesis Using Macromonomer of Example 6

In this example 8, a copolymer designated IE P2 was synthesized. The copolymer backbone was made of 60 VAc/40 Si10PrVi by weight. A three-neck, 250-mL round-bottom flask was equipped with a condenser, a thermocouple, and a Y-shaped glass adapter for two polyethylene feed lines. The flask was first charged with an egg-shaped Teflon-coated magnetic stir bar and 13.50 g of ethyl acetate. The flask was placed onto an Opti-chem™ hotplate stirrer, and the temperature was raised to 65° C. A nitrogen blanket was applied to remove the entrained air, and the agitation rate was at 300 rpm. In a separate 60-mL glass jar, 10.80 g of VAc and 7.20 g of the macromonomer prepared according to Example 6 were charged in order and allowed to form a homogeneous monomer mixture. The cofeed initiator was 0.24 g of Trigonox 125-C75 in 6.75 g of EtOAc. When the temperature of the reactor reached 65° C., 3.60 g of the homogeneous monomer mixture was charged into the reactor. A few minutes later, the rest of the homogeneous monomer mixture and the cofeed initiator started to be metered at the rates of 0.12 g/min and 0.0583 g/min, respectively. The targeted feed time was 120 min. When the monomer feed was completed, 1.80 g of EtOAc was added into the monomer syringe and rinsed into the reactor. The batch was held at 65° C. for 15 min. Then two chemical chases of 0.36 g of Trigonox 125-C75 in 1.35 g of EtOAc were metered in at a rate of 0.057 g/min over 30 min with a 15-min hold in between. The batch was held for another 30 min before being quenched by air cooling. The final product was analyzed by ¹H NMR spectroscopy for the monomer conversion, GPC for the molecular weight and dispersity, and DSC for glass-transition temperatures (T_{g}).

Characterization of the final product was a copolymer solid: 40 wt %. Residual VAc was 240 ppm by ¹H NMR spectroscopy. The residual macromonomer (of Example 6) content was below the detection limit (typically deemed as 1000 ppm) by ¹H NMR spectroscopy. The Mₙ, M_{w}, and dispersity of the copolymer were 16.0 kg/mol, 54.8 kg/mol, and 3.42, respectively (relative to PS standards). T_{g} was 3.0° C. by DSC using the 2^{nd} heating at a rate of 20° C./min.

Additional copolymers were made according to the procedure of Example 8, but varying the amounts monomers in the monomer mixture and the type of macromonomer. The amounts and selections are shown below in Table 2.

### Example 9—Copolymer Synthesis Using Macromonomer of Example 6

In this example 9, a copolymer designated IE P5 was prepared as described below. The copolymer backbone was made of 50 VAc/40 Si10PrVi/10 MDO by weight. A three-neck, 250-mL round-bottom flask was equipped with a condenser, a thermocouple, and a Y-shaped glass adapter for two polyethylene feed lines. The flask was first charged with an egg-shaped Teflon-coated magnetic stir bar and 13.50 g of ethyl acetate. The flask was placed onto an Opti-chem™ hotplate stirrer, and the temperature was raised to 65° C. A nitrogen blanket was applied to remove the entrained air, and the agitation rate was at 300 rpm. In a separate 60-mL glass jar, 9.00 g of VAc, 7.20 g of Si10PrVi (the macromonomer prepared according to Example 6), and 1.80 g of MDO were charged in order and allowed to form a homogeneous monomer mixture. The cofeed initiator was 0.24 g of Trigonox 125-C75 in 6.75 g of EtOAc. When the temperature of the reactor reached 65° C., 3.60 g of the homogeneous monomer mixture was charged into the reactor. A few minutes later, the rest of the homogeneous monomer mixture and the co-feed initiator started to be metered via syringe pumps at the rate of 0.12 g/min and 0.0583 g/min, respectively. The targeted feed time was 120 min. When the monomer feed was completed, 1.80 g of EtOAc was added into the monomer syringe and rinsed into the reactor. The batch was held at 65° C. for 15 min. Then two chemical chases of 0.36 g of Trigonox 125-C75 in 1.35 g of EtOAc were metered in at a rate of 0.057 g/min over 30 min with a 15-min hold in between. Then the batch was held for another 30 min before being quenched by air cooling.

Characterization of the final product: solid: 40 wt %. The residual VAc, Si10PrVi, and MDO content were below the detection limit by ¹H NMR spectroscopy (typically deemed as 1000 ppm). The Mₙ, M_{w}, and dispersity were 6.1 kg/mol, 35.9 kg/mol, and 5.88, respectively (relative to PS standards). T_{g} was −19.4° C. by DSC using the 2^{nd} heating at a rate of 20° C./min.

Additional copolymers were made according to the procedure of Example 9, but varying the amounts monomers in the monomer mixture and the type of monomers. The amounts and selections are shown below in Table 2.

### Example 10—Copolymer Synthesis Using Macromonomer of Example 6

In this example 10, a copolymer designated IE P4 was prepared as described below. The copolymer backbone was made of 56% VAc/40% Si10PrVi/4% AA by weight. A three-neck, 250-mL round-bottom flask was equipped with a condenser, a thermocouple, and a Y-shaped glass adapter for two polyethylene feed lines. The flask was first charged with an Teflon-coated magnetic stir bar and a mixture of 10.13 g of ethyl acetate and 3.38 g of ethanol. The flask was placed onto an Opti-chem™ hotplate stirrer, and the temperature was raised to 65° C. A nitrogen blanket was applied to remove the entrained air, and the agitation rate was at 300 rpm. In a separate 60-mL glass jar, 10.08 g of VAc, 7.20 g of Si10PrVi (the macromonomer prepared according to Example 6), and 0.72 g of AA were charged in order and allowed to form a homogeneous monomer mixture. The cofeed initiator was 0.24 g of Trigonox 125-C75 in a mixture of 5.06 g of ethyl acetate and 1.69 g of ethanol. When the temperature of the reactor reached 65° C., 3.60 g of the homogeneous monomer mixture was charged into the reactor. A few minutes later, the rest of the homogeneous monomer mixture and the co-feed initiator started to be metered via syringe pumps at the rate of 0.12 g/min and 0.0583 g/min, respectively. The targeted feed time was 120 min. The reaction mixture became opaque during feeding. A first charge of 1.69 g of ethanol was added to the reactor about 45 min after the start of feeds, and a second charge of 1.69 g of ethanol was added to the reactor about 90 min after the start of feeds. When the monomer feed was completed, a mixture of 1.35 g of ethyl acetate and 0.45 g of ethanol was added into the monomer syringe and rinsed into the reactor. The batch was held at 65° C. for 15 min. Then two chemical chases of 0.48 g of Trigonox 125-C75 in a mixture of 1.35 g of ethyl acetate and 0.45 g of ethanol were metered in at a rate of 0.076 g/min over 30 min with a 15-min hold in between. Then the batch was held for another 30 min before being quenched by air cooling.

### Example 11—Synthesis of MCR-V21-Aldehyde

In this Example 11, hydroformylation of MCR-V21 was performed, as follows: In a nitrogen filled glovebox, Rh(acac)(CO)₂ (0.0023 g), Ligand 1 (0.0147 g) and toluene (3.0 g) were added into a vial with a magnetic stir bar. The mixture was stirred at RT on a stir plate until a homogeneous catalyst solution was formed. The catalyst solution was transferred to an air-tight syringe with a metal valve and subsequently removed from the glove box. In a ventilated fume hood, MCR-V21 (90.0 g, 0.015 mol) was loaded to a 300 mL Parr-reactor. The reactor was sealed and pressurized with nitrogen up to 100 psig (689 kPa) via the dip-tube and was carefully relieved through a valve connected to the headspace. The pressure/vent cycle with nitrogen was repeated three times. Pressure testing was subsequently performed by pressurizing the reactor with nitrogen to up to 300 psig (2086 kPa). After the pressure was released, the catalyst solution was added to the reactor via the sample loading port. The reactor was pressurized with syngas to 100 psig (689 kPa) and then vented for three times prior to being pressurized to 20 psig (138 kPa) below the desired pressure via the dip-tube. The reaction temperature was set to 70° C. The heater and agitation were turned on. The reaction was run at 100 psig (689 kPa) syngas pressure. 99.5% conversion of vinyl groups was observed after 5.5 hours reaction time as monitored by ¹H NMR. The reactor was vented and purged with nitrogen for three times before the product (MCR-V21-Aldehyde) was collected and evaporated under vacuum.

### Example 12—Synthesis of MCR-V21-Acid

In this Example 12, oxidation of MCR-V21-aldehyde (prepared as described in Synthesis Example 1t was performed as follows. MCR-V21-aldehyde (90 g) which contained approximately 5 wt % toluene was loaded to a one-neck round bottom flask equipped with a PTFE coated stir bar and a septa cap. The liquid was stirred on a magnetic stir plate at 1150 rpm as air was sparged subsurface through a needle. The reaction mixture was analyzed by ¹H NMR until complete. The reaction was stopped after 24 hours and the MCR-V21-acid product was collected as a clear orange liquid.

### Example 13—Synthesis of MCR-V21-Vinyl Ester

In this Example 13, a linear vinylester-functional polydimethylsiloxane was prepared using carboxy-functional polydimethylsiloxane prepared from the hydroformylation of MCR-V21 from Gelest as the starting material. To a three-neck round bottom flask equipped with a thermometer, a condenser with a bubbler on top and a rubber septum was used for this synthesis. A heating mantle with a J-Kem controller was used for controlling the heating. A magnetic stir bar, MCR-V21-Aldehyde(75.8 g) prepared as described above in Example 11, and vinyl acetate (26.1 g, 0.302 mol) were added to the reactor. Nitrogen was bubbled into the mixture subsurface through a needle under vigorous stirring for 5 minutes. Palladium acetate (0.172 g, 0.000766 mol), phenanthroline (0.21 g, 0.0012 mol) and MEHQ (0.023 g, −0.00018 mol) were added via the septa port. This mixture was stirred for 10 minutes before the reaction mixture was heated to 60° C. overnight. After the reaction was done, materials were transferred to a round bottom flask and evaporated on a rotary evaporator with a bath temperature of 40° C. (500-10 mbar). 200 mL of hexane was added to the flask. The mixture was filtered through a disposable filter with 7 g of silica. The filtrate was concentrated under a rotary evaporator, affording 72.1 g brown liquid.

### Example 14—Synthesis of Si4-Aldehyde

In this Example 14, hydroformylation of 1,1,1,5,5,5-hexamethyl-3-((trimethylsilyl)oxy)-3-vinyltrisiloxane (Si4Vi) was performed, as follows: In a nitrogen filled glovebox, Rh(acac)(CO)₂ (0.0023 g), Ligand 1 (0.0147 g) and toluene (3.0 g) were added into a vial with a magnetic stir bar. The mixture was stirred at RT on a stir plate until a homogeneous catalyst solution was formed. The catalyst solution was transferred to an air-tight syringe with a metal valve and subsequently removed from the glove box. In a ventilated fume hood, Si4Vi (177.8 g, 0.055 mol) was loaded to a 300 mL Parr-reactor. The reactor was sealed and pressurized with nitrogen up to 100 psig (689 kPa) via the dip-tube and was carefully relieved through a valve connected to the headspace. The pressure/vent cycle with nitrogen was repeated three times. Pressure testing was subsequently performed by pressurizing the reactor with nitrogen to up to 300 psig (2086 kPa). After the pressure was released, the catalyst solution was added to the reactor via the sample loading port. The reactor was pressurized with syngas to 100 psig (689 kPa) and then vented for three times prior to being pressurized to 20 psig (138 kPa) below the desired pressure via the dip-tube. The reaction temperature was set to 70° C. The heater and agitation were turned on. The reaction was run at 100 psig (689 kPa) syngas pressure. 99.5% conversion of vinyl groups was observed after 5.5 hours reaction time as monitored by ¹H NMR. The reactor was vented and purged with nitrogen for three times before the product 3-(1,1,1,5,5,5-hexamethyl-3-((trimethylsilyl)oxy)trisiloxan-3-yl)propanal was collected and evaporated under vacuum.

### Example 15—Synthesis of Si4-Acid

In this Example 15, oxidation of 3-(1,1,1,5,5,5-hexamethyl-3-((trimethylsilyl)oxy)trisiloxan-3-yl)propanal (Si4-aldehyde) (prepared as described in Example 14 was performed as follows. Si4-aldehyde (90 g) which contained approximately 5 wt % toluene was loaded to a one-neck round bottom flask equipped with a PTFE coated stir bar and a septa cap. The liquid was stirred on a magnetic stir plate at 1150 rpm as air was sparged subsurface through a needle. The reaction mixture was analyzed by ¹H NMR until complete. The reaction was stopped after 24 hours and the 3-(1,1,1,5,5,5-hexamethyl-3-((trimethylsilyl)oxy)trisiloxan-3-yl)propanoic acid (Si4-Acid) product was collected as a clear orange liquid.

### Example 16—Synthesis of Si4-Vinyl Ester

In this Example 16, vinyl 3-(1,1,1,5,5,5-hexamethyl-3-((trimethylsilyl)oxy)trisiloxan-3-yl)propanoate (Si4-VE) was prepared using Si4-Acid as the starting material. To a three-neck round bottom flask equipped with a thermometer, a condenser with a bubbler on top and a rubber septum was used for this synthesis. A heating mantle with a J-Kem controller was used for controlling the heating. A magnetic stir bar, Si4-Acid (27.8 g, 0.0759 mol) and vinyl acetate (136.1 g, 1.58 mol) were added to the reactor. Nitrogen was bubbled into the mixture subsurface through a needle under vigorous stirring for 5 minutes. Palladium acetate (0.214 g, 0.000955 mol) and phenanthroline (0.214 g, 0.0012 mol) and MEHQ (0.036 g, 0.00029 mol) were added via the septa port. This mixture was stirred for 10 minutes before the reaction mixture was heated to 60° C. overnight. After the reaction was done, materials were transferred to a round bottom flask and evaporated on a rotary evaporator with a bath temperature of 40° C. (500-10 mbar). 200 mL of hexane was added to the flask. The mixture was filtered through a disposable filter with 7 g of silica. The filtrate was concentrated under a rotary evaporator, affording 27.6 g clear oil.

Characterization of the final product: solid: 35.4 w˜t %. The conversion of VAc was 99.8% by 1H NMR spectroscopy, and the residual Si10PrVi and AA content were below the detection limit by 1H NMR spectroscopy (typically deemed as 1000 ppm). The Mn, Mw, and dispersity were 4.5 kg/mol, 18.6 kg/mol, and 4.17, respectively (relative to PS standards). Tg was 6.6° C. by DSC using the 2nd heating at a rate of 20° C./min.

TABLE 2
COMPOSITION OF THE COMPARATIVE
AND INVENTIVE POLYMER EXAMPLES

| | | | | Theoretical |
|---|---|---|---|---|
| | | | Solid | biodegradation |
| | (Co)Polymer description/ | | content | content of polymers |
| Examples | composition | Carrier | (wt %) | (wt %) |
| CE P1 | 100% VAc | EtOAc | 40 | 100 |
| CE P2 | DOWSIL ™ FA4012 IDD | Isododecane | 43.5 | 20 |
| CE P3 | DOWSIL ™ FA4004 IDD | Isododecane | 40 | 19 |
| IE P1 | 70% VAc/30% Si10PrVi | EtOAc | 40 | 72 |
| IE P2 | 60% VAc/40% Si10PrVi | EtOAc | 40 | 62 |
| IE P3 | 50% VAc/50% Si10PrVi | EtOAc | 40 | 53 |
| IE P4 | 56% VAc/40% Si10PrVi/ | 66 EtOAc/34 | 35.4 | 58 |
| | 4% AA | EtOH | | |
| IE P5 | 50% VAc/40% Si10PrVi/ | EtOAc | 40 | 62 |
| | 10% MDO | | | |
| IE P6 | 70% VAc/30% | EtOAc | 40 | 71 |
| | Si10HepVi | | | |
| IE P7 | 60% VAc/40% | EtOAc | 40 | 62 |
| | Si10HepVi | | | |
| IE P8 | 50% VAc/50% | EtOAc | 40 | 52 |
| | Si10HepVi | | | |
| IE P9 | 90% VAc/10% Si10PrVi | EtOAc | 40 | 91 |
| IE P10 | 80% VAc/20% Si10PrVi | EtOAc | 40 | 81 |
| IE P11 | 80% VAc/20% | EtOAc | 40 | 81 |
| | Si10HepVi | | | |

In Table 2, above, solid content refers to the amount of (co)polymer dissolved in the carrier. The theoretical biodegradation contents of the (co)polymers in Table 2 were calculated using Equation 1 below.

$$biodeg ( polymer ) = {\sum }_{i} biodeg ( {monomer}_{i} ) \times w ( {monomer}_{i} ) \qquad ( Equation l )$$

In Equation 1, biodeg(monomerᵢ) is the theoretical biodegradation content of a constituent monomerᵢ, and w(mnonomnerᵢ) is the weight percentage of mnonomnerᵢ in the polymer. The biodeg(nionomnerᵢ) values are listed in Table 3. In the calculation, polyvinyl esters undergo hydrolysis and afford poly(vinyl alcohol) (PVOH) as the water-soluble polymeric backbone and small-molecule carboxylic acids from hydrolysis of the side chain groups. PVOH and organic alkanoic acids such as acetic acid are biodegradable. However, for the purposes of this calculation, siloxane-based carboxylic acids are assumed to be non-biodegradable. For (meth)acrylate polymers, hydrolysis affords poly(meth)acrylic acid as the backbone and the corresponding alcohols from the side chain groups. Poly(meth)acrylic acids are assumed to be non-biodegradable if the Mw is above 2000 g/mol. The small-molecule organic alkanols such as methanol are biodegradable. Siloxane-functionalized alcohols are assumed to be non-biodegradable.
CE P1 is a VAc homopolymer, which has 100% theoretical biodegradation content.
CE P2 and CE P3 are commercially available silicone-acrylate copolymers, which have 19% to 20% biodegradation content.
The silicone-vinylester copolymers prepared as described herein (IE P1 to IE P11) each have >50% biodegradation content, where the exact biodegradation content for each copolymer depends on the amount of polyorganosiloxane and vinylester content of each copolymer.

TABLE 3
THEORETICAL BIODEGRADATION CONTENT
OF MONOMERIC REPEAT UNITS

| | Overall | MW of the | Theoretical |
|---|---|---|---|
| | MW | biodeg portion | biodegradation content |
| Monomer | (g/mol) | (g/mol) | (wt %) |
| VAc | 86.09 | 86.09 | 100 |
| Si10PrVi | 839.69 | 43.05 | 5.1 |
| Si10HepVi | 895.79 | 43.05 | 4.8 |
| MDO | 114.14 | 114.14 | 100 |
| AA | 72.06 | 72.06 | 0 |

The polymer (comparative example CE P1) and the copolymers prepared as described above in Table 2 were characterized using the test methods described below. The number average molecular weight (Mₙ), weight average molecular weight (M_{w}), and dispersity (calculated by M_{w}/Mₙ) measured by GPC, and the glass transition temperature (T_{g}) measured by DSC are reported in Table 4

TABLE 4
MOLECULAR AND THERMAL CHARACTERISTICS
OF COMPARATIVE AND INVENTIVE
POLYMER EXAMPLES.

| Polymer | Conversion | Mₙ | M_{w} | Disper- | T_{g} |
|---|---|---|---|---|---|
| Examples | (by ¹H NMR) | (kg/mol) | (kg/mol) | sity | (° C.) |
| CE P1 | VAc: Quantitative | 19.3 | 84.3 | 4.37 | 16.7 |
| IE P1 | VAc: quantitative, | 19.3 | 93.2 | 4.82 | 14.6 |
| | Si10PrVi: | | | | |
| | quantitative | | | | |
| IE P2 | VAc: 99.9%, | 16.2 | 56.0 | 3.47 | 3.0 |
| | Si10PrVi: | | | | |
| | quantitative | | | | |
| IE P3 | VAc: 99.6%, | 16.0 | 54.8 | 3.42 | 3.0 |
| | Si10PrVi: 99.2% | | | | |
| IE P4 | VAc: 99.8%, | 4.5 | 18.6 | 4.17 | 6.6 |
| | Si10PrVi: | | | | |
| | quantitative, AA: | | | | |
| | quantitative | | | | |
| IE P5 | VAc: quantitative, | 6.1 | 35.9 | 5.88 | −19.4 |
| | Si10PrVi: | | | | |
| | quantitative, MDO: | | | | |
| | quantitative | | | | |
| IE P6 | VAc: 99.8%, | 15.6 | 69.0 | 4.42 | 18.2 |
| | Si10HepVi: | | | | |
| | quantitative | | | | |
| IE P7 | VAc: 99.6%, | 15.0 | 67.6 | 4.50 | 13.0 |
| | Si10HepVi: | | | | |
| | quantitative | | | | |
| IE P8 | VAc: 99.9%, | 13.8 | 63.1 | 4.56 | 5.0 |
| | Si10HepVi: | | | | |
| | quantitative | | | | |
| IE P9 | VAc: 99.9%, | 13.0 | 51.3 | 3.95 | 24.7 |
| | Si10PrVi: | | | | |
| | quantitative | | | | |
| IE P10 | VAc: 99.9%, | 17.4 | 73.8 | 4.25 | 22.0 |
| | Si10PrVi: | | | | |
| | quantitative | | | | |
| IE P11 | VAc: 99.8%, | 16.4 | 75.8 | 4.63 | 22.7 |
| | Si10HepVi: | | | | |
| | quantitative | | | | |

The data in Table 4 show that silicone-vinylester copolymers with varying amounts of starting materials M1) and M2) can be prepared under the conditions of examples 8 and 9. Furthermore, copolymers including starting material M3) were also successfully prepared (in IE P4 and IE P5).

The samples of polymer (CE P1) and copolymers (IF P1 to IE P11) prepared as described in Examples 7 to 10 were analyzed for water contact angle and sebum contact angle according to the test methods below. Comparative commercially available silicone-acrylate copolymer compositions were also tested (CE P2 and CE P3) The contact angles (CA) of neat films to evaluate the intrinsic water and sebum repellency of the (co)polymer compositions are shown in Table 5. Higher contact angle values indicate higher repellency of either water or sebum, as noted.

TABLE 5
WATER & SEBUM CONTACT ANGLE DATA FOR NEAT FILMS PREPARED
FROM THE COMPARATIVE AND INVENTIVE EXAMPLES

| | | Water | Sebum | Water | Sebum |
|---|---|---|---|---|---|
| | | CA @ | CA @ | CA @ | CA @ |
| | (Co)Polymer | t= 0 s | t = 0 s | t = 200 s | t = 200 s |
| Sample | composition | (°) | (°) | (°) | (°) |
| CE P1 | 100% VAc polymer | 57.8 ± | 16.6 ± | 54.4 ± | 6.8 ± |
| | | 2.1 | 1 | 3.7 | 1.6 |
| CE P2 | DOWSIL ™ FA4012 IDD | 102.2 ± | 45.2 ± | 100.5 ± | 42.7 ± |
| | | 1.6 | 3.5 | 1.6 | 5.6 |
| CE P3 | DOWSIL ™ FA4004 IDD | 100.8 ± | 50.2 ± | 99.9 ± | 50.3 ± |
| | | 1 | 1.6 | 1.5 | 0.8 |
| IE P1 | 70% VAc/30% Si10PrVi | 102.3 ± | 61 ± | 98.4 ± | 60.9 ± |
| | copolymer | 2.1 | 1.4 | 2.4 | 1.4 |
| IE P2 | 60% VAc/40% Si10PrVi | 107.9 ± | 61.4 ± | 106.4 ± | 61 ± |
| | copolymer | 2.3 | 4.9 | 2.9 | 4.2 |
| IE P3 | 50% VAc/50% Si10PrVi | 108.7 ± | 61.6 ± | 106.7 ± | 59.5 ± |
| | copolymer | 4.7 | 5 | 1.7 | 4.3 |
| IE P4 | 56% VAc/40% Si10PrVi/ | 111.5 ± | 64.6 ± | 103.1 ± | 62.8 ± |
| | 4% AA** copolymer | 2 | 1.2 | 1.9 | 1.1 |
| IE P5 | 50% VAc/40% Si10PrVi/ | 112.5 ± | 71.1 ± | 99.4 ± | 61.8 ± |
| | 10% MDO copolymer | 2 | 0.7 | 1.3 | 0.8 |
| IE P6 | 70% VAc/30% Si10HepVi* | 89 ± | 46.6 ± | 85.9 ± | 46.4 ± |
| | copolymer | 1.4 | 3.1 | 2.7 | 3.3 |
| IE P7 | 60% VAc/40% Si10HepVi* | 98.6 ± | 48.6 ± | 94.9 ± | 47.7 ± |
| | copolymer | 8.1 | 1.6 | 6.3 | 2.4 |
| IE P8 | 50% VAc/50% Si10HepVi* | 99.5 ± | 50.6 ± | 96.2 ± | 49.1 ± |
| | copolymer | 6.8 | 0.8 | 8.5 | 1.3 |
| IE P9 | 90% VAc/10% Si10PrVi | 84.4 ± | 39.4 ± | 79.2 ± | 40.7 ± |
| | copolymer | 1.1 | 3.1 | 1.4 | 1.9 |
| IE P10 | 80% VAc/20% Si10PrVi | 87.3 ± | 43.2 ± | 83.9 ± | 42.1 ± |
| | copolymer | 1.1 | 3.6 | 1.2 | 5.2 |
| IE P11 | 80% VAc/20% Si10HepVi* | 83.8 ± | 45.5 ± | 79.6 ± | 45.2 ± |
| | copolymer | 0.8 | 1.3 | 1.4 | 1.6 |

Five measurements were carried out on each film. The average and standard deviation are reported in Table 5. In Table 5, the ‘*’ denotes that the macromonomer prepared as described above in Example 3 was used to prepare the copolymner sample instead of the mnacromnonomner prepared as described above in Example 6. The ‘**’ denotes that 4% acrylic acid (AA) was used instead of 10% MDO as M3) the additional monomer in preparation of copolymer IE P4 according to the method of Example 9. Comparative Examples 2 and 3 (CE2 and CE3) in the table above showed commercially available silicone-acrylate copolymers.

Copolymers IE P1 to IE P11 demonstrated that copolymers could be successfully synthesized with varying levels of M1) the vinylester-functional siloxane macromonomer and M2) the vinylester of an aliphatic fatty acid using the process described herein. Samples IE P1 to IE P11 showed a benefit over CE P1 in that water contact angle and sebum contact angle remained consistent over 200 s, whereas sample CE P1 showed a significant decrease in sebum contact angle over time (i.e., sebum contact angle decreased from 16.6 to 6.8 after 200 seconds). This attribute suggests excellent hydrophobicity and low water absorption of the inventive copolymers, and thus holds potential of better durability for formulated personal-care products after application to keratinous surface, e.g., skin. In contrast, copolymers containing 10% to 50% of the vinylester-functional siloxane macromonomer prepared as described herein had more consistent sebum contact angles over time, thereby suggesting that a benefit of the copolymers described herein is that the copolymers may provide sebum resistance that can be maintained with time.

In addition, copolymers IE P1 to IE P8 showed a benefit over the vinylacetate homopolymer in sample CE P1. CE P1 showed initial water contact angles at 57.8°. Copolymers IE P1 to IE P8 showed initial water contact angles at 90-110°; these examples showed essentially the same water contact angles after 200 seconds. These results demonstrated the excellent water repellency of copolymers IE P1 to IE P8. Surprisingly, certain copolymers (e.g., IE P2, IE P3, IE P4, and IE P5) showed higher water contact angles than commercially available silicone-acrylate compositions (CE P5 & P6).

Similar composition/performance relationships were observed in sebum contact angle measurements. The comparative polymer CE P1 showed an initial sebum contact angle of 16.6⁰. In comparison, the copolymers IE P1 to IE P11 all showed higher initial sebum contact angles than CE P1. These copolymers further showed an order of magnitude higher sebum contact angle after 200 seconds than the sebum contact angle of 6.8° of polymer CE P1. These results demonstrated the excellent sebum repellency of the copolymers prepared as described herein. Surprisingly, certain copolymers (e.g., IE P1, IE P2, IE P3, IE P4, and IE P5) also showed higher sebum contact angles than comparative commercially available silicone-acrylate compositions (CE 2 and CE 3).

### Preparation of Cosmetic Foundation Formulations

The copolymers described in Table 2 were tested in a cosmetic foundation formulation. Foundations for color cosmetics were prepared using the formulation shown in Table 6 and according to the following general procedure. Starting materials 1-5 were first added to a plastic cup and homogenized inside a FlackTeck speedmixer at a centrifugal rate of 2000 rpm for 1 min. Starting materials 6 and 7 were added to the same cup and the mixture was homogenized at the same centrifugal rate for an additional 1 min to afford Phase A. Starting materials 8-11 were prepared in a separate vessel and homogenized with an overhead mixer at 500-800 rpm until a uniform mixture of Phase B was obtained. Then the Phase B mixture was slowly added to the Phase A mixture while mixing with an overhead mixer operating at 500-800 rpm. Phase C was subsequently introduced to the mixture of Phase A & B, under the same mixing conditions, until a uniform mixture was obtained. Phase D was finally added, under the same mixing conditions, until a uniform mixture was obtained.

TABLE 6
FORMULATIONS FOR FOUNDATIONS CONTAINING
SI-ACRYLATE OR SI-VINYL ESTER
IIYBRID FILM FORMERS

| Starting | | Weight |
|---|---|---|
| Materials | INCI | (%) |
| Phase A | | |
| 1 | Isododecane | 12 |
| 2 | Iron Oxide (CI77499) (and) dimethicone | 0.07 |
| 3 | Iron Oxide (CI77491) (and) dimethicone | 0.25 |
| 4 | Iron Oxide (CI77492) (and) dimethicone | 1.09 |
| 5 | Titanium dioxide (and) dimethicone | 5.81 |
| 6 | Caprylyl Methicone | 3.28 |
| 7 | Lauryl PEG-10 Tris(trimethylsiloxy)silylethyl | 6 |
| | Dimethicone | |
| Phase B | | |
| 8 | Water | 47.7 |
| 9 | Sodium chloride | 1 |
| 10 | Glycerin | 5 |
| 11 | Phenoxyethanol (and) ethylhexyglycerin | 1 |
| Phase C | (Co)Polymer described above | 5 (based |
| | | on active) |
| Phase D | Water | q.s. 100 |

The foundation formulations described in Table 6 were cast into thin films and dried. Three tests were carried out as follows:
Visual assessment of film quality
Water and sebum contact angles
Rub-off resistance

TABLE 7
FOUNDATION FILM QUALITY

| Foundation | | Visual Film |
|---|---|---|
| Examples | (Co)Polymer | Quality Rating |
| CE F1 | Control, no polymer was added | 3 |
| CE F2 | 100% VAc (CE P1) | 1 |
| CE F3 | 80% VAc/20% Si10PrVi (IE P10) | 1 |
| CE F4 | DOWSIL ™ FA4012 IDD (CE P2) | 4 |
| CE F5 | DOWSIL ™ FA4004 IDD (CE P3) | 5 |
| IE F1 | 60% VAc/40% Si10PrVi (IE P2) | 5 |
| IE F2 | 50% VAc/50% Si10PrVi (IE P3) | 5 |
| IE F3 | 60% VAc/40% Si10HepVi (IE P7) | 5 |
| IE F4 | 50% VAc/50% Si10HepVi (IE P8) | 5 |

The test methods are described below. Results are shown in Table 7.

### Film Rating Criteria:

5—fine & smooth, shiny, no apparent defects
4—largely fine & smooth, but slightly uneven thickness or color
3—largely fine & smooth, some pinholes or uneven color
2—no cracks, continuous polymer film, no grains, but noticeable pinholes or uneven color
1—no cracks, continuous polymer film, but grainy, noticeable pinholes or uneven color
0—cracks, does not form continuous film

Certain copolymers prepared as described herein provided excellent film quality in the foundation formulations tested under the conditions described above. These data suggest excellent film-forming capability of the copolymers, particularly when the copolymer contained more than 20% of the vinylester-functional siloxane macromonomer described herein.

Water contact angle and sebum contact angle were also tested on the foundation films prepared as described above from the foundation formulations in Table 6. The results are shown in Table 8.

TABLE 8
WATER AND SEBUM CONTACT ANGLES OF FOUNDATION FILMS

| | | Water | Sebum | Water | Sebum |
|---|---|---|---|---|---|
| | | CA @ | CA @ | CA @ | CA @ |
| Foundation | (Co)polymer | t = 0 s | t = 0 s | t = 200 s | t = 200 s |
| Examples | composition | (°) | (°) | (°) | (°) |
| CE F1 | Control, no polymer | 109.3 ± 1.2 | 51 ± 2.2 | 98.2 ± 1 | 22.2 ± 3.8 |
| | was added | | | | |
| CE F2 | 100 VAc (CE P1) | 121.5 ± 3.9 | 48.1 ± 1.7 | 106.6 ± 2.1 | 21 ± 1.2 |
| CE F3 | 80 VAc/20 Si10PrVi | 110.1 ± 4.7 | 48.5 ± 1 | 98.7 ± 7.2 | 20.5 ± 0.6 |
| | (IE P10) | | | | |
| CE F4 | DOWSIL ™ FA4012 | 99.7 ± 4.1 | 41.6 ± 0.8 | 95.5 ± 3.7 | 33 ± 2 |
| | IDD (CE P2) | | | | |
| CE F5 | DOWSIL ™ FA4004 | 101 ± 2.9 | 54.3 ± 0.4 | 100.7 ± 0.5 | 52.9 ± 0.6 |
| | IDD (CE P3) | | | | |
| IE F1 | 60 VAc/40 Si10PrVi | 105.2 ± 0.9 | 45.8 ± 1.1 | 93.6 ± 1.3 | 23.8 ± 1.1 |
| | (IE P2) | | | | |
| IE F2 | 50 VAc/50 Si10PrVi | 115.2 ± 4.9 | 51 ± 1 | 104.8 ± 5.4 | 18.2 ± 0.6 |
| | (IE P3) | | | | |
| IE F3 | 60 VAc/40 | 106.9 ± 4.6 | 64.4 ± 1.9 | 102.8 ± 2.6 | 40.2 ± 1.2 |
| | Si10HepVi (IE P7) | | | | |
| IE F4 | 50 VAc/50 | 98.6 ± 0.8 | 65.8 ± 6.3 | 89.9 ± 2.3 | 22.3 ± 0.6 |
| | Si10HepVi (IE P8) | | | | |

Five measurements were carried out on each film. The average and standard deviation are reported. The data in Table 8 indicated IE F3 and IE F4 showed higher initial sebum contact angle than all other samples tested. Without wishing to be bound by theory, it is thought that this result is due to the heptanoate group from the macromonomer used to prepare the copolymer, which may provide a further benefit to sebum resistance relative the propanoate group used to prepare other copolymers in the foundation formulations tested.

Rub off was also evaluated on the foundation films prepared as described above, according to the test methods below. Results are in Table 9.

TABLE 9
COLOR CHANGE AFTER RUB-OFF
TESTS OF FOUNDATION FILMS

| Foundation Examples | (Co)Polymer composition | ΔE |
|---|---|---|
| CE F1 | Control, no polymer was added | 7.1 ± 1.7 |
| CE F2 | 100 VAc (CE P1) | 4.0 ± 0.5 |
| CE F3 | 80 VAc/20 Si10PrVi (IE P10) | 4.5 ± 2.0 |
| CE F4 | DOWSIL ™ FA4012 IDD (CE P2) | 1.4 ± 0.3 |
| CE F5 | DOWSIL ™ FA4004 IDD (CE P3) | 3.6 ± 1.3 |
| IE F1 | 60 VAc/40 Si10PrVi (IE P2) | 4.5 ± 1.0 |
| IE F2 | 50 VAc/50 Si10PrVi (IE P3) | 6.8 ± 1.7 |
| IE F3 | 60 VAc/40 Si10HepVi (IE P7) | 3.6 ± 0.9 |
| IE F4 | 50 VAc/50 Si10HepVi (IE P8) | 3.3 ± 1.6 |

In Table 9, ten measurements were carried out on each film. The average and one standard deviation are reported. The lower the delta E value, the better the rub off resistance. The results in Table 9 suggest that the copolymers prepared as described herein have comparable or better rub off resistance to a vinylester homopolymer (CE P1 in foundation example CE F2) and to the commercial silicone-acrylate compositions (CE P2 and CE P3 in samples CE F3 and CE F4, respectively).

### Preparation of Oil-In-Water Sunscreen Formulations

Oil-in-water (O/W) sunscreen formulations were then prepared using the copolymers as described above in Table 2 according to the procedure below, using the types and amounts of starting materials shown below in Table 10. Phase A ingredients were combined in a suitable size vessel, and mixed while being heated to 75° C. to afford a uniform mixture. In a separate vessel, Phase B ingredients were combined, and mixed while being heated to 75° C. until full dissolution. Phase B was added into Phase A while being mixed at a moderate speed. After all Phase B was added, the batch was maintained at 75° C. while being mixed at a moderate speed. Phase C was added into batch and mixed until a uniform mixture was obtained. Phase D was pre-heated to 75° C. and added into batch and mixed until a uniform mixture was obtained. Ethyl acetate in each original copolymer solution had been removed by distillation, and the resulting dried copolymers were reconstituted in either isododecane or C12-15 alkyl benzoate as 40 wt % solutions. The batch was then cooled and mixed at a moderate speed. When batch temperature was below 45° C., Phase E was added and mixed at a moderate speed until the batch reached room temperature.

TABLE 10
FORMULATIONS FOR OIL-IN-WATER SUNSCREEN EXAMPLES

| | CE | IE | IE | IE | IE | CE |
|---|---|---|---|---|---|---|
| | S(O/ | S(O/ | S(O/ | S(O/ | S(O/ | S(O/ |
| Sunscreen | W) | W) | W) | W) | W) | W) |
| Formulation | 2 | 1 | 2 | 3 | 4 | 1 |
| Ingredients | wt. % | wt. % | wt. % | wt. % | wt. % | wt. % |
| Phase A | | | | | | |
| DI Water | 61.50 | 61.10 | 61.10 | 61.10 | 61.10 | 66.10 |
| Disodium EDTA | 0.10 | 0.10 | 0.10 | 0.10 | 0.10 | 0.10 |
| Acrylates/Vinyl | 2.00 | 2.00 | 2.00 | 2.00 | 2.00 | 2.00 |
| Neodecanoate | | | | | | |
| Crosspolymer | | | | | | |
| 1,3-Propanediol | 2.00 | 2.00 | 2.00 | 2.00 | 2.00 | 2.00 |
| Phase B | | | | | | |
| PEG 40 Stearate | 1.00 | 1.00 | 1.00 | 1.00 | 1.00 | 1.00 |
| Glyceryl Stearate | 3.00 | 3.00 | 3.00 | 3.00 | 3.00 | 3.00 |
| Cetearyl | 2.00 | 2.00 | 2.00 | 2.00 | 2.00 | 2.00 |
| Alcohol (and) | | | | | | |
| Cetereth-20 | | | | | | |
| Avobenzone | 3.00 | 3.00 | 3.00 | 3.00 | 3.00 | 3.00 |
| Octisalate | 5.00 | 5.00 | 5.00 | 5.00 | 5.00 | 5.00 |
| Octocrylene | 5.00 | 5.00 | 5.00 | 5.00 | 5.00 | 5.00 |
| Homosalate | 5.00 | 5.00 | 5.00 | 5.00 | 5.00 | 5.00 |
| Isododecane | 5.00 | 5.00 | 5.00 | | 5.00 | 5.00 |
| C12-15 Alkyl | | | | 5.00 | | |
| Benzoate | | | | | | |
| Phase C | | | | | | |
| Sodium | 0.30 | 0.30 | 0.30 | 0.30 | 0.30 | 0.30 |
| Hydroxide, | | | | | | |
| 20% sol. | | | | | | |
| Phase D | | | | | | |
| CE P2 | 4.60 | | | | | |
| DOWSIL ™ | | | | | | |
| FA 4012 | | | | | | |
| ID | | | | | | |
| IE P2 in | | 5.00 | | | | |
| Isododecane | | | | | | |
| IE P3 in | | | 5.00 | | | |
| Isododecane | | | | | | |
| IE P4 in | | | | 5.00 | | |
| C12-15 Alkyl | | | | | | |
| Benzoate | | | | | | |
| IE P5 in | | | | | 5.00 | |
| Isododecane | | | | | | |
| Phase E | | | | | | |
| Phenoxyethanol | 0.50 | 0.50 | 0.50 | 0.50 | 0.50 | 0.50 |
| Total | 100.00 | 100.00 | 100.00 | 100.00 | 100.00 | 100.00 |

### Procedure of Preparation of Water-In-Oil Sunscreen Formulations:

Water-in-oil (WIG) sunscreen formulations were then prepared using the copolymers as described above in Table 2 according to the procedure below, using the types and amounts of starting materials shown below in Table 11. Phase A ingredients were combined and mixed until a uniform mixture was obtained. In a suitable size vessel, Phase B ingredients were combined and mixed at a moderate speed. Phase B was then heated to 75° C. and mixed until all ingredients were dissolved and a uniform mixture was obtained. Ethyl acetate in the original silicone-vinylester copolymer solutions had been removed by distillation, and the dried copolymers were reconstituted in either isododecane or C12-15 alkyl benzoate as 40 wt % solutions. Under homogenization at 4000 rpm, Phase A was slowly added into Phase B. Homogenization was continued at 4000 rpm for 3 minutes after all Phase A was added. The batch was transferred to be stirred under an overhead mixer at a moderate speed. When batch temperature below 45° C., Phase C was added and mixed at moderate speed until batch reached room temperature.

TABLE 11
FORMULATIONS FOR WATER-IN-OIL SUNSCREEN EXAMPLES

| | CE | IE | IE | IE | IE | CE |
|---|---|---|---|---|---|---|
| | S(W/ | S(W/ | S(W/ | S(W/ | S(W/ | S(W/ |
| | O) | O) | O) | O) | O) | O) |
| Example ID | 2 | 1 | 2 | 3 | 4 | 1 |
| Ingredients | wt. % | wt. % | wt. % | wt. % | wt. % | wt. % |
| Phase A | | | | | | |
| DI Water | 54.40 | 54.00 | 54.00 | 54.00 | 54.00 | 59.00 |
| Sodium Chloride | 0.50 | 0.50 | 0.50 | 0.50 | 0.50 | 0.50 |
| 1,3-Propanediol | 3.00 | 3.00 | 3.00 | 3.00 | 3.00 | 3.00 |
| Phase B | | | | | | |
| Dimethicone | 6.00 | 6.00 | 6.00 | 6.00 | 6.00 | 6.00 |
| (and) PEG/PPG- | | | | | | |
| 18/18 | | | | | | |
| Dimethicone | | | | | | |
| Cetyl | 3.00 | 3.00 | 3.00 | 3.00 | 3.00 | 3.00 |
| Dimethicone | | | | | | |
| Avobenzone | 3.00 | 3.00 | 3.00 | 3.00 | 3.00 | 3.00 |
| Octisalate | 5.00 | 5.00 | 5.00 | 5.00 | 5.00 | 5.00 |
| Octocrylene | 5.00 | 5.00 | 5.00 | 5.00 | 5.00 | 5.00 |
| Homosalate | 5.00 | 5.00 | 5.00 | 5.00 | 5.00 | 5.00 |
| Isododecane | 1.00 | 1.00 | 1.00 | 1.00 | 1.00 | 1.00 |
| C12-15 Alkyl | 1.00 | 1.00 | 1.00 | 1.00 | 1.00 | 1.00 |
| Benzoate | | | | | | |
| Dimethicone | 8.00 | 8.00 | 8.00 | 8.00 | 8.00 | 8.00 |
| 2 cst | | | | | | |
| CE P2 DOWSIL | 4.60 | | | | | |
| FA 4012 IDD | | | | | | |
| IE P2 in | | 5.00 | | | | |
| Isododecane | | | | | | |
| IE P3 in | | | 5.00 | | | |
| Isododecane | | | | | | |
| IE P4 in | | | | 5.00 | | |
| C12-15 Alkyl | | | | | | |
| IE P5 in | | | | | 5.00 | |
| Isododecane | | | | | | |
| Phase C | | | | | | |
| Phenoxyethanol | 0.50 | 0.50 | 0.50 | 0.50 | 0.50 | 0.50 |
| Total | 100.00 | 100.00 | 100.00 | 100.00 | 100.00 | 100.00 |

Both O/W and W/G sunscreen formulations were cast uniformly into thin films and dried. The initial SPF values were measured to evaluate any SPF boosting effect. Three measurements were carried out on each formulation. The average and one standard deviation are reported. The in vitro SPF results of O/W sunscreen formulations are shown below in Table 12. The in vitro SPF results of W/O sunscreen formulations are shown below in Table 13.

TABLE 12
IN VITRO SPF RESULTS FOR OIL-IN-
WATER TYPE SUNSCREEN FORMULATIONS

| Sunscreen (Oil in water) | (Co)Polymer | | |
|---|---|---|---|
| Examples | Example | (Co)Polymer composition | in vitro SPF |
| CE S(O/W)1 | blank | N/A | 9.52 ± 0.87 |
| CE S(O/W)2 | CE P2 | FA4012 (CE P2) | 8.82 ± 0.44 |
| IE S(O/W)1 | IE P2 | 60% VAc/40% Si10PrVi (IE P2) | 12.39 ± 1.11 |
| IE S(O/W)2 | IE P3 | 50% VAc/50% Si10PrVi (IE P3) | 10.33 ± 0.69 |
| IE S(O/W)3 | IE P4 | 56% VAc/40% Si10PrVi/4% AA | 29.43 ± 3.11 |
| | | (IE P4) | |
| IE S(O/W)4 | IE P5 | 50% VAc/40% Si10PrVi/10% MDO | 12.48 ± 0.51 |
| | | (IE P5) | |

All of the copolymers prepared as described herein and tested in the O/W sunscreen had an SPF boosting effect. The in vitro SPF value was higher than the control with no copolymer and the comparative O/W formulation containing a commercially available silicone-acrylate copolymer.

TABLE 13
IN VITRO SPF RESULTS FOR WATER-IN-
OIL TYPE SUNSCREEN FORMULATIONS

| ICD Sunscreen | (Co)Polymer | | |
|---|---|---|---|
| (W/O) Example | Example | (Co)Polymer composition | in vitro SPF |
| CE S(W/O)1 | Blank | N/A | 4.44 ± 0.34 |
| CE S(W/O)2 | CE P2 | FA4012 | 5.97 ± 0.51 |
| IE S(W/O)1 | IE P2 | 60% VAc/40% Si10PrVi (IE P2) | 4.78 ± 0.39 |
| IE S(W/O)2 | IE P3 | 50% VAc/50% Si10PrVi (IE P3) | 7.37 ± 0.18 |
| IE S(W/O)3 | IE P4 | 56% VAc/40% Si10PrVi/4% AA (IE P4) | 4.52 ± 0.24 |
| IE S(W/O)4 | IE P5 | 50% VAc/40% Si10PrVi/10% MDO (IE P5) | 6.56 ± 0.35 |

Water-in-oil sunscreen formulations containing copolymer IE P3 and copolymer IE P5 showed some SPF boosting.

### Test Methods Used in the Examples

### ¹H Nuclear Magnetic Resonance (NMR) Spectroscopy

A small amount (50-100 mg) of a solution polymer sample was dissolved in about 1.0 mL of deuterated chloroform, and analyzed on a Bruker AVANCE III HD 500 spectrometer equipped with a 5 mm PRODIGY CryoProbe. Quantitative ¹H spectra were acquired with the zg30 pulse sequence, a relaxation delay of 60 seconds, and 64 scans. The spectra were analyzed using MestReNova software (version 12) from Mestrelab Research. Residual monomers were quantified by comparing the signals from the vinyl region of the monomers to those from the solvent.

### Gel-Permeation Chromatography (GPC)

Solution polymers were diluted in tetrahydrofuran to a concentration of about 2.0 mg/mL, and analyzed on a GPC consisting of an Agilent 1260 Infinity II Model isocratic pump and an Agilent 1260 Infinity II Refractive Index detector. Tetrahydrofuran was the mobile phase, the elution rate was 1.0 mL/min, and the separation was enabled by two PLgel Mixed A columns (300×7.5 mm inner diameter) and a guard column (50×7.5 mm inner diameter). Ten narrow polystyrene standards in the range of 580 g/mol to 6,800,000 g/mol were used to construct a 1^{st}-order fit calibration curve. Agilent GPC/SEC software Version A.02.01 were used to process the data.

### Glass Transition Temperature T_{g})

A small amount of a solution polymer was transferred to an aluminum pan. The solution polymer was first dried at ambient temperature, and then at 60° C. under house vacuum until constant mass was achieved. The dried polymer mass was typically 3 to 10 mg. The aluminum pan was hermetically sealed and analyzed on a Q1000 differential scanning calorimeter from TA Instruments. Two heating scans were applied between −90° C. and 150° C. at a rate of 20° C./min. The values from the second scan was reported.

### Preparation of Polymer Neat Films and Contact Angle Measurement

Thin films of the solution polymers were prepared manually using a 6 mil doctor blade on LENETA black plastic charts. The films were dried inside a fume hood at 23° C. for at least 72 hours.

Contact angle data were acquired from the surface of each film using both water and sebum on a Kruss DSA100 instrument. Data were acquired as quickly as possible (0 seconds) and after approximately 200 seconds on the same drop. Five drops were applied to the same piece of film, and the average and standard deviation values are reported.

### Rub-Off Test of Cured Foundation Films

Foundation samples were each coated a black vinyl chart (available from Leneta) using a doctor blade film applicator with the gap set at 6 mils (0.1524 mm) and allowed to dry at 22° C. for at least 48 hours. The color reading of each sample was then measured by colorimeter (Ocean Optics).

The wear resistance of the deposited film of color cosmetic formulations was characterized by the change (ΔE) before and after abrasion with a pre-cut bath towel (55 mm×45 mm). The bath towel was fixed to a moving robotic part that moves back and forth periodically at a constant speed. The film was abraded by the bath towel by 3 wear cycles under a pressure of approximately 600 Pa. Readings were taken from ten point on each deposited film.

### In Vitro SPF Measurement

in vitro SPF measurement was conducted using a LABSHPERE UV 2000S Spectrometer. First, about 0.0325 g of each formulation was weighed on a HELIOPLATE HD6 PMMA substrate. The formulation was then spread uniformly using the index finger covered with rubber finger cot. The sample was allowed to dry at ambient conditions for 20-30 minutes. The UV absorbance between 290 nm and 400 nm was measured at 9 locations on the dried sample. The selection of 9 locations was guided by the positioning marks on the instrument sample stage assembly. An in vitro SPF value was generated and recorded at the end of each measurement. Each formulation was measured 3 times, and the in vitro SPF value of each formulation was calculated as an averaged value of the 3 measurement.

### INDUSTRIAL APPLICABILITY

The examples above show that M1) the vinylester-functional siloxane macromonomer (macromonomer) was successfully synthesized and copolymerized with M2) the vinylester of the aliphatic fatty acid to form the silicone-vinylester copolymer (copolymer). The copolymer was successfully synthesized with varying amounts of the macromonomer and the vinylester of the aliphatic fatty acid. Additional monomers, M3), were also successfully included in some copolymer samples. The copolymer is useful in different personal care compositions, for example, cosmetic foundations and sunscreens. The desired properties of the copolymer, such as biodegradation potential, or water resistance and/or sebum resistance in different personal care formulations, can be tailored by varying the types and amounts of: M1) the macromonomer and M2) the vinylester of the aliphatic fatty acid, and by adding M3) the optional additional monomer.

### Definitions and Usage of Terms

All amounts, ratios, and percentages herein are by weight, unless otherwise indicated. The amounts of all starting materials in a composition total 100% by weight. The SUMMARY and ABSTRACT are hereby incorporated by reference. The articles ‘a’, ‘an’, and ‘the’ each refer to one or more, unless otherwise indicated by the context of specification. The singular includes the plural unless otherwise indicated. The transitional phrases “comprising”, “consisting essentially of”, and “consisting of” are used as described in the Manual of Patent Examining Procedure Ninth Edition, Revision 08.2017, Last Revised January 2018 at section § 2111.03 I., II., and III. The abbreviations used herein have the definitions in Table Z.

TABLE Z
Abbreviations

| Abbreviation | Definitions |
|---|---|
| acac | acetyl acetonate |
| ° C. | degrees Celsius |
| cc | cubic centimeters |
| D | Difunctional siloxy unit, trimethylsiloxy unit of formula Me₂SiO_{2/2} |
| D^{al} | Difunctional siloxy unit, allylmethylsiloxy unit of formula |
| | (Allyl)(Me)SiO_{2/2} |
| D^{hex} | Difunctional siloxy unit, hexenylmethylsiloxy unit of formula |
| | (Hex)(Me)SiO_{2/2} |
| D^{Pr-ald} | Difunctional siloxy unit, propylaldehyde, methylsiloxy unit of formula (Pr- |
| | ald)(Me)SiO_{2/2} |
| D^{vi} | Difunctional siloxy unit, vinylmethylsiloxy unit of formula (Vi)(Me)SiO_{2/2} |
| DP | degree of polymerization |
| DSC | Dow Silicones Corporation of Midland, Michigan, USA |
| Et | ethyl |
| FTIR | Fourier transform infra-red |
| g | gram |
| GPC | gel permeation chromatography |
| h or hr | hour |
| Hex | hexenyl |
| iPr | isopropyl |
| kPa | kiloPascals |
| M | Monofunctional siloxy unit, trimethylsiloxy unit of formula R₃SiO_{1/2} or |
| | Me₃SiO_{1/2} |
| M^{al} | Monofunctional siloxy unit, allyldimethylsiloxy unit of formula |
| | (Allyl)(Me₂)SiO_{1/2} |
| M^{hex} | Monofunctional siloxy unit, hexenyldimethylsiloxy unit of formula |
| | (Hex)(Me₂)SiO_{1/2} |
| M^{Pr-ald} | Monofunctional siloxy unit, propyladehyde, dimethylsiloxy unit of formula |
| | (Pr-ald)(Me₂)SiO_{1/2} |
| M^{vi} | Monofunctional siloxy unit, vinyldimethylsiloxy unit of formula |
| | (Vi)(Me₂)SiO_{1/2} |
| Me | methyl |
| mg | milligram |
| min | minute |
| mL | milliliter |
| mm | millimeter |
| Mmol | millimole |
| Mn | number average molecular weight measured by GPC |
| Mw | weight average molecular weight measured by GPC |
| mPa · s | milliPascal seconds |
| NBD | norbornadiene |
| NMR | nuclear magnetic resonance |
| PDI | Polydispersity index (calculated as Mw/Mn) |
| PDMS | polydimethylsiloxane |
| Ph | phenyl |
| ppm | parts per million by weight |
| Pr | propyl |
| Pr-ald | propyl-aldehyde |
| psi | pounds per square inch |
| PTFE | polytetrafluoroethylene |
| Q | tetrafunctional siloxy unit of formula SiO_{4/2} |
| RPM or rpm | revolutions per minute |
| RT | room temperature of 25 ± 5° C. |
| TDCC | The Dow Chemical Company of Midland, Michigan, USA |
| THF | tetrahydrofuran |
| μ or u | micro |
| μm | micrometer |
| Vi | vinyl |

The following test methods were used herein. ²⁹Si NMR: Alkenyl content of the alkenyl-functional polyorganosiloxanes described herein can be measured by the technique described in “The Analytical Chemistry of Silicones” ed. A. Lee Smith, Vol. 112 in Chemical Analysis, John Wiley & Sons, Inc. (1991). Viscosity: Viscosity may be measured at 25° C. at 0.1 to 50 RPM on a Brookfield DV-III cone & plate viscometer with #CP-52 spindle, e.g., for polymers (such as certain alkenyl-functional polyorganosiloxanes, aldehyde-functional polyorganosiloxanes, carboxy-functional polyorganosiloxanes and vinylester-functional siloxane macromonomers) with viscosity of 120 mPa-s to 250,000 mPa·s. One skilled in the art would recognize that as viscosity increases, rotation rate decreases and would be able to select appropriate spindle and rotation rate.

1. A method for making a silicone-vinylester-functional copolymer, wherein the method comprises:
preparing M1) a vinylester-functional siloxane macromonomer by a process comprising
combining, under conditions to conduct transvinylation reaction, starting materials comprising
(I) a carboxy-functional polyorganosiloxane,
(J) an alkenyl acetate,
(K) a transvinylation catalyst, and
optionally (L) a third solvent, and
optionally (M) an inhibitor,
thereby producing a transvinylation reaction product comprising (M1) the vinylester-functional siloxane macromonomer;
optionally recovering (M1) the vinylester-functional siloxane macromonomer;
I) combining, under conditions to conduct free radical polymerization, starting materials comprising:
M1) the vinylester-functional siloxane macromonomer,
M2) a vinyl ester of an aliphatic fatty acid,
optionally M3) an additional alkenyl-functional monomer,
N) a free radical initiator; and
optionally O) an organic carrier;
thereby forming a reaction mixture comprising the silicone-vinylester-functional copolymer;
II) quenching the reaction mixture after step I);
optionally III) recovering the silicone-vinylester-functional copolymer from the reaction mixture;
optionally IV) dissolving the silicone-vinylester-functional copolymer in a simple alcohol; and
optionally V)) performing a solvent exchange after II), or after step III) or after step IV), when step III) or step IV) is present.

2. The method of claim 1, where
M1) the vinylester-functional siloxane macromonomer is used in an amount of 1 weight % to 99 weight %;
M2) the vinyl ester of the aliphatic fatty acid is used in an amount of 1 weight % to 99 weight %;
M3) the additional alkenyl-functional monomer is used in an amount of 0 to 20 weight %;
each based on combined weights of M1), M2), and M3).

3. The method of claim 1, where M1) vinylester-functional siloxane macromonomer has formula 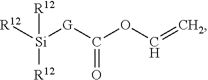
where G is a divalent hydrocarbon group free of aliphatic unsaturation, each R¹² is independently selected from —OSi(R¹⁴)₃ and R¹³, where each R¹³ is a monovalent hydrocarbon group; where each R¹⁴ is selected from R¹³, —OSi(R¹⁵)₃, and —[OSiR¹³₂]ᵢᵢOSiR¹³₃; where each R¹⁵ is selected from R¹³, —OSi(R¹⁶)₃, and —[OSiR¹³₂]ᵢᵢOSiR¹³₃; where each R¹⁶ is selected from R¹³ and —[OSiR¹³₂]ᵢᵢOSiR¹³₃; and where each subscript ii independently has a value such that 0≤ii≤100, with the proviso that at least two of R¹² are —OSi(R¹⁴)₃ and the vinylester-functional polyorganosiloxane has 4 to 16 silicon atoms per molecule.

4. The method of claim 3, where M1) the vinylester-functional siloxane macromonomer comprises formula: 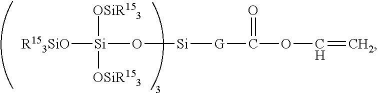
where G and R¹⁵ are as described above.

5. The method of claim 3, where M1) the vinylester-functional siloxane macromonomer comprises formula: 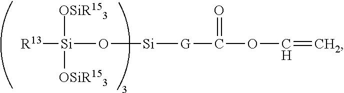
where G, R¹³, and R¹⁵ are as described above.

6. The method of claim 3, where M1) the vinylester-functional siloxane macromonomer comprises formula: 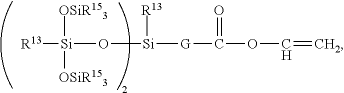
where G, R¹³, and R¹⁵ are as described above.

7. The method of claim 3, where M1) the vinylester functional siloxane macromonomer comprises one or more of:

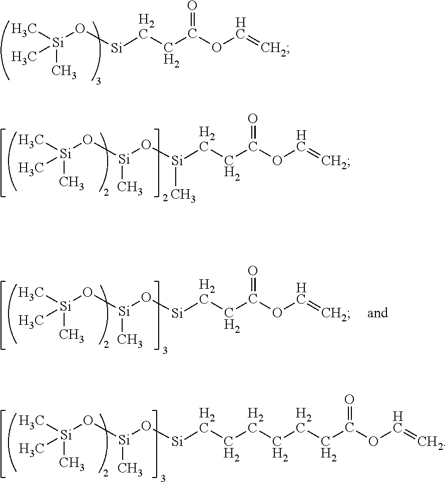

8. (canceled)

9. The method of claim 1, where (I) the carboxy-functional polyorganosiloxane is prepared by a process comprising:
combining, under conditions to conduct oxidation reaction, starting materials comprising
(E) an aldehyde-functional polyorganosiloxane, and
(F) an oxygen source,
thereby forming an oxidation reaction product comprising (I) the carboxy-functional polyorganosiloxane; and
optionally recovering (I) the carboxy-functional polyorganosiloxane.

10. The method of claim 9, where (E) the aldehyde-functional polyorganosiloxane is prepared by a process comprising:
combining, under conditions to catalyze hydroformylation reaction, starting materials comprising
(A) a gas comprising hydrogen and carbon monoxide,
(B) an alkenyl-functional polyorganosiloxane,
(C) a rhodium/bisphosphite ligand complex catalyst, where the bisphosphite ligand has formula 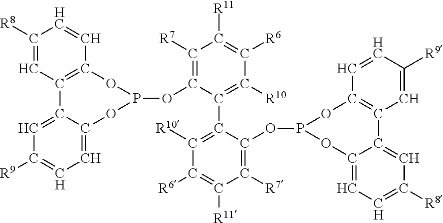
where
R⁶ and R^{6′} are each independently selected from the group consisting of hydrogen, an alkyl group of 1 to 20 carbon atoms, a cyano group, a halogen group, and an alkoxy group of 1 to 20 carbon atoms;
R⁷ and R^{7′} are each independently selected from the group consisting of an alkyl group of 3 to 20 carbon atoms, and a group of formula —SiR¹⁷₃, where each R¹⁷ is an independently selected monovalent hydrocarbon group of 1 to 20 carbon atoms;
R⁸, R^{8′}, R^{9′} and R^{9′} are each independently selected from the group consisting of hydrogen, an alkyl group, a cyano group, a halogen group, and an alkoxy group, and
R¹⁰ R^{10′}, R¹¹, and R^{11′} are each independently selected from the group consisting of hydrogen or and alkyl group; thereby forming a hydroformylation reaction product comprising the aldehyde-functional polyorganosiloxane, and
optionally (D) a solvent;
thereby forming a hydroformylation reaction product comprising (E) the aldehyde-functional polyorganosiloxne; and
optionally recovering (E) the aldehyde-functional branched polyorganosiloxne oligomer.

11. The method of claim 1, where M2) the vinyl ester of the aliphatic fatty acid has formula: 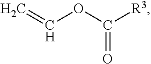
where R³ is hydrogen or an alkyl group of 1 to 14 carbon atoms.

12. The method of claim 1, where M2) the vinyl ester of the aliphatic fatty acid comprises vinyl acetate.

13. The method of claim 1, where
M1) the vinylester-functional siloxane macromonomer is used in an amount of 30 weight % to 60 weight %,
M2) the vinyl ester of the aliphatic fatty acid is used in an amount of 40 weight % to 70 weight %, and
M3) the additional alkenyl-functional monomer is used in an amount of 0 to 20 weight %;
each based on based on combined weights of M1), M2), and M3).

14. (canceled)

15. (canceled)

16. The method of claim 1, where M1) vinylester-functional siloxane macromonomer comprises unit formula:
(R⁴₃SiO_{1/2})ₐ(R^{VE}R⁴₂SiO_{1/2})_{b}(R⁴₂SiO_{2/2})_{c}(R^{VE}R⁴SiO_{2/2})_{d}, where R^{VE} is 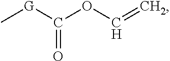
where G is a divalent hydrocarbon group free of aliphatic unsaturation, each R⁴ is independently selected from the group consisting of an alkyl group of 1 to 18 carbon atoms and an aryl group of 6 to 18 carbon atoms, and subscripts a, b, c, and d represent average numbers of each unit in the formula and have values such that subscript a is 0, 1, or 2; subscript b is 0, 1, or 2, subscript c≥0, subscript d≥0, with the provisos that a quantity (b+d)≥1, a quantity (a+b)=2, and a quantity (a+b+c+d)≥2.

17. The method of claim 16, where M1) the vinylester-functional siloxane macromonomer has formula: 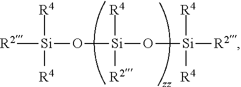
where each R^{2′″} is independently selected from the group consisting of R⁴ and R^{VE}, with the proviso that at least one R^{2′″} per molecule is R^{VE}, each R⁴ is independently selected from the group consisting of an alkyl group of 1 to 18 carbon atoms and an aryl group of 6 to 18 carbon atom; each R^{VE} is 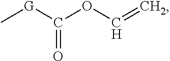
where G is a divalent hydrocarbon group free of aliphatic unsaturation; and subscript zz=a quantity (c+d).

18. The method of claim 16, where M1) the vinylester-functional siloxane macromonomer has formula: 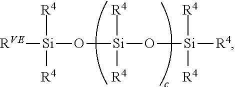
where each R⁴ is independently selected from the group consisting of an alkyl group of 1 to 18 carbon atoms and an aryl group of 6 to 18 carbon atom; each R^{VE} is 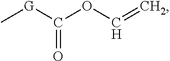
where G is a divalent hydrocarbon group free of aliphatic unsaturation; and subscript c is 0 to 2,000.

19. The method of claim 16, where
M1) the vinylester-functional siloxane macromonomer is used in an amount of 30 weight % to 60 weight %,
M2) the vinyl ester of the aliphatic fatty acid is used in an amount of 40 weight % to 70 weight %, and
M3) the additional alkenyl-functional monomer is used in an amount of 0 to 20 weight %;
each based on based on combined weights of M1), M2), and M3).

20. (canceled)

21. (canceled)

22. A method for preparing a personal care composition, wherein the method comprises adding the silicone-vinylester copolymer prepared by the method of claim 19 into the personal care composition.

23. A method for preparing a personal care composition, wherein the method comprises adding the silicone-vinylester copolymer prepared by the method of claim 13 into the personal care composition.
